(19) United States
(12) Patent Application Publication
(10) Pub. No.: US 2023/0084382 A1
(43) Pub. Date: Mar. 16, 2023

(54) TREATMENT OF CANCER WITH A COMBINATION OF AN ANTIBODY THAT BINDS LGR5 AND EGFR AND A TOPOISOMERASE I INHIBITOR

(71) Applicant: Merus N.V., Utrecht (NL)

(72) Inventors: Mark THROSBY, Utrecht (NL); Ernestro Isaac WASSERMAN, Utrecht (NL)

(21) Appl. No.: 17/632,181

(22) PCT Filed: Aug. 19, 2020
(86) PCT No.: PCT/NL2020/050517

§ 371 Date: Feb. 1, 2022

(30) Foreign Application Priority Data
Aug. 19, 2019 (EP) 19192327.5

(51) Int. Cl. A61K 39/395; C07K 16/28; C07K 16/30; A61K 31/4745; A61P 35/00

(52) U.S. Cl. CPC A61K 39/3955; C07K 16/2863; C07K 16/30; A61K 31/4745; A61P 35/00; C07K 2317/56; C07K 2317/732; A61K 2039/545

(57) ABSTRACT

The invention describes antibodies or functional parts, derivatives and/or analogues thereof that comprise a variable domain that binds an extracellular part of EGFR and a variable domain that binds an extracellular part of LGR5 for use in the treatment of cancer wherein the antibody or functional part, derivative and/or analogue thereof is administered with a topoisomerase I inhibitor.

### CROSS-REFERENCE TO RELATED APPLICATIONS

This application is the U.S. national phase of PCT/NL2020/050517, filed Aug. 19, 2020, which claims priority to European Application No. 19192327.5, filed Aug. 19, 2019.

### REFERENCE TO SEQUENCE LISTING SUBMITTED ELECTRONICALLY VIA EFS-WEB

The Sequence Listing submitted Jan. 31, 2022, as a text file named “4096_0400001_Seqlisting_ST25.txt”, created on Jan. 31, 2022, and having a size of 106,315 bytes, is hereby incorporated by reference pursuant to 37 C.F.R. § 1.52(e)(5).

The invention relates to means and methods in the treatment of cancer. The invention in particular relates to a method of treating a cancer in a subject with a combination of an antibody that binds LGR5 and EGFR and a topoisomerase inhibitor. The invention further relates to the combination for use in such methods and to the combination for use in the manufacture of a medicament for the treatment of gastrointestinal cancer.

Colorectal cancer (CRC) is the third most common cancer in the world. While some new treatments have been advanced in CRC, many have failed clinical testing and metastatic CRC is still largely incurable. The current standard-of-care for advanced CRC in the clinics includes chemotherapy regimens which block essential functions in cancer cells and kill dividing cells.

Accumulating evidence suggests that cancer growth and re-growth after treatment-induced remission is caused by populations of cancer stem cells which evade chemotherapy treatment. Without being bound by theory it is believed that maintenance of these stem cells is thought to be regulated by the WNT signaling pathway.

Without being bound by theory it is believed that a second oncogenic pathway in CRC, thought to be responsible for enhanced cancer cell proliferation and apoptosis evasion—is the EGFR (Epidermal Growth Factor Receptor) pathway. Several anti-EGFR drugs have demonstrated certain levels of efficacy for targeted therapy of metastatic CRC (mCRC). However, due to heterogeneity of CRC, oncogenic mutations in the downstream KRAS gene confer resistance to anti-EGFR therapies (˜40% of all mCRC patients), half of patients with wild type KRAS have innate resistance to anti-EGFR therapies, and most of the patients of which the cancers are sensitive to anti-EGFR treatment show resistant cancer later on.

In its Biclonics® antibody program, Merus has developed multispecific antibodies that target EGFR and LGR5 (Leucine-rich repeat containing G protein-coupled receptor), a stem cell marker in WNT signaling pathway. The efficacy of such multispecific antibodies has been assessed in vitro and in vivo using patient-derived CRC organoids and mice PDX models, respectively. Multispecific antibodies that target EGFR and LGR5 were shown to inhibit tumor growth. The potency of such inhibitory antibodies was shown to be correlated with the levels of LGR5 RNA expression by cells from the cancer.

The present invention shows that a combination therapy comprising the administration of a multispecific antibody that targets EGFR and LGR5 in combination with a topoisomerase I inhibitor is surprisingly effective when compared to the effect of the multispecific antibody or topoisomerase antibody separately. Such a combination therapy can inhibit metastasis and/or re-growth of the tumor after treatment-induced remission in CRC patients and achieve longer remissions.

### SUMMARY OF THE INVENTION

The invention provides an antibody or functional part, derivative and/or analogue thereof that comprises a variable domain that binds an extracellular part of EGFR and a variable domain that binds an extracellular part of LGR5 for use in the treatment of cancer wherein the antibody or functional part, derivative and/or analogue thereof is administered with a topoisomerase I inhibitor. The cancer is preferably colorectal, pulmonary, gastrointestinal or ovarian cancer, preferably colorectal cancer. The antibody or functional part, derivative and/or analogue thereof and the topoisomerase I inhibitor are preferably administered to the subject concurrently.

In one aspect the antibody or functional part, derivative and/or analogue thereof is administered to the subject prior to the topoisomerase I inhibitor.

The variable domain that binds an extracellular part of EGFR may comprise the amino acid sequence of VH chain MF3755 as depicted in FIG. 8; or the amino acid sequence of VH chain MF3755 as depicted in FIG. 8 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH. The variable domain that binds an extracellular part of LGR5 may comprise the amino acid sequence of VH chain MF5816 as depicted in FIG. 8; or the amino acid sequence of VH chain MF5816 as depicted in FIG. 8 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH. The antibody preferably comprises both of said variable domains.

The variable domain that binds LGR5 preferably binds an epitope that is located within amino acid residues 21-118 of the human LGR5 sequence depicted in FIG. 1. In one embodiment the amino acid residues at positions 43, 44, 46, 67, 90, and 91 of human LGR5 are involved in the binding of the provided LGR5 binding variable domain to LGR5. The LGR5 binding variable domain preferably binds less to an LGR5 protein comprising one or more of the amino acid residue variations selected from 43A, 44A, 46A, 67A, 90A, and 91A.

The variable domain that binds EGFR preferably binds an epitope that is located within amino acid residues 420-480 of the human EGFR sequence depicted in FIG. 2. In one embodiment the amino acid residues at positions 1462, G465, K489, 1491, N493 and C499 of human EGFR are involved in the binding of the provided EGFR binding variable domain to EGFR. The EGFR binding variable domain preferably binds less to an EGFR protein comprising one or more of the amino acid residue substitutions selected from I462A, G465A, K489A, I491A, N493A and C499A.

The provided antibody or functional part, derivative and/or analogue thereof as described herein preferably comprises both an LGR5 binding variable domain with the epitope binding characteristic as described herein above and an EGFR binding variable domain with the epitope binding characteristic as described herein above.

In one embodiment the topoisomerase I inhibitor is camptothecin or a derivative thereof. In another, preferred embodiment the topoisomerase I inhibitor is irinotecan or topotecan.

The antibody is preferably an ADCC inducing antibody. In an embodiment the antibody is an ADCC enhanced antibody. In one embodiment the antibody is afucosylated.

The invention also provides a method for inhibiting proliferation of a cell that expresses EGFR and LGR5 in a system permissive for proliferation of the cell, the method comprising providing the system with a topoisomerase I inhibitor and with an antibody or functional part, derivative and/or analogue thereof that comprises a variable domain that binds an extracellular part of EGFR and a variable domain that binds an extracellular part of LGR5.

Further provided is a method of treatment of cancer in a subject comprising administering simultaneously or sequentially a topoisomerase I inhibitor and an antibody or functional part, derivative and/or analogue thereof that comprises a variable domain that binds an extracellular part of EGFR and a variable domain that binds an extracellular part of LGR5 to the subject in need thereof.

The cancer is preferably colorectal, pulmonary, gastrointestinal or ovarian cancer. In a preferred embodiment the cancer is colorectal cancer.

The invention also provides a pharmaceutical composition comprising an antibody or functional part, derivative and/or analogue thereof that comprises a variable domain that binds an extracellular part of EGFR and a variable domain that binds an extracellular part of LGR5; and a topoisomerase I inhibitor. The antibody or functional part, derivative and/or analogue thereof and the topoisomerase I inhibitor can be provided in a single formulation. The antibody or functional part, derivative and/or analogue thereof and the topoisomerase I inhibitor can also be provided in separate formulations. When provided in separate formulations both medicaments can be administered simultaneously or sequentially.

Further provided is a kit comprising an antibody or functional part, derivative and/or analogue thereof that comprises a variable domain that binds an extracellular part of EGFR and a variable domain that binds an extracellular part of LGR5; a topoisomerase I inhibitor and instructions for use of the antibody and the topoisomerase I inhibitor in the treatment as described herein.

Further provided is an antibody or functional part, derivative and/or analogue thereof that comprises a variable domain that binds an extracellular part of EGFR and a variable domain that binds an extracellular part of LGR5 for use in the treatment of gastrointestinal cancer in a subject, wherein the antibody is administered simultaneously, separately or sequentially with a topoisomerase I inhibitor.

In one aspect the invention provides an antibody or functional part, derivative and/or analogue thereof that comprises a variable domain that binds an extracellular part of EGFR and a variable domain that binds an extracellular part of LGR5 for use in the manufacture of a medicament for the treatment of cancer in a subject, wherein the antibody is administered simultaneously, separately or sequentially with a topoisomerase I inhibitor. The treatment is preferably against colorectal, pulmonary, gastrointestinal or ovarian cancer, preferably against colorectal cancer.

Also provided herein is a product comprising an antibody or functional part, derivative and/or analogue thereof that comprises a variable domain that binds an extracellular part of EGFR and a variable domain that binds an extracellular part of LGR5 and a topoisomerase I inhibitor as a combined preparation for simultaneous, separate or sequential use in the treatment of gastrointestinal cancer in a subject.

### DETAILED DESCRIPTION OF THE INVENTION

In order that the present description may be more readily understood, certain terms are first defined. Additional definitions are set forth throughout the detailed description. Unless stated otherwise, all technical and scientific terms used herein have the same meaning as commonly understood by one of ordinary skill in the art, and conventional methods of immunology, protein chemistry, biochemistry, recombinant DNA techniques and pharmacology are employed.

As used herein, the singular forms “a”, “an” and “the” include plural referents unless the context clearly dictates otherwise. Use of the term “including” as well as other forms, such as “include”, “includes”, and “included”, is not limiting.

The term “antibody” as used herein means a proteinaceous molecule belonging to the immunoglobulin class of proteins, containing one or more domains that bind an epitope on an antigen, where such domains are or derived from or share sequence homology with the variable region of an antibody. Antibodies are typically made of basic structural units each with two heavy chains and two light chains. Antibodies for therapeutic use are preferably as close to natural antibodies of the subject to be treated as possible (for instance human antibodies for human subjects). An antibody according to the present invention is not limited to any particular format or method of producing it.

A “bispecific antibody” is an antibody as described herein wherein one domain of the antibody binds to a first antigen whereas a second domain of the antibody binds to a second antigen, wherein said first and second antigens are not identical. The term “bispecific antibody” also encompasses antibodies wherein one heavy chain variable region/light chain variable region (VH/VL) combination binds a first epitope on an antigen and a second VH/VL combination that binds a second epitope. The term further includes antibodies wherein VH is capable of specifically recognizing a first antigen and the VL, paired with the VH in an immunoglobulin variable region, is capable of specifically recognizing a second antigen. The resulting VH/VL pair will bind either antigen 1 or antigen 2. Such so called “two-in-one antibodies”, described in for instance WO 2008/027236, WO 2010/108127 and Schaefer et al (Cancer Cell 20, 472-486, October 2011). A bispecific antibody according to the present invention is not limited to any particular bispecific format or method of producing it.

The term ‘common light chain’ as used herein refers to the two light chains (or the VL part thereof) in the bispecific antibody. The two light chains (or the VL part thereof) may be identical or have some amino acid sequence differences while the binding specificity of the full length antibody is not affected. The terms ‘common light chain’, ‘common VL’, ‘single light chain’, ‘single VL’, with or without the addition of the term ‘rearranged’ are all used herein interchangeably. “Common” also refers to functional equivalents of the light chain of which the amino acid sequence is not identical. Many variants of said light chain exist wherein mutations (deletions, substitutions, insertions and/or additions) are present that do not influence the formation of functional binding regions. The light chain of the present invention can also be a light chain as specified herein, having from 0 to 10, preferably from 0 to 5 amino acid insertions, deletions, substitutions, additions or a combination thereof. It is for instance within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like. The term ‘full length IgG’ or ‘full length antibody’ according to the invention is defined as comprising an essentially complete IgG, which however does not necessarily have all functions of an intact IgG. For the avoidance of doubt, a full length IgG contains two heavy and two light chains. Each chain contains constant (C) and variable (V) regions, which can be broken down into domains designated CH1, CH2, CH3, VH, and CL, VL. An IgG antibody binds to antigen via the variable region domains contained in the Fab portion, and after binding can interact with molecules and cells of the immune system through the constant domains, mostly through the Fc portion. Full length antibodies according to the invention encompass IgG molecules wherein mutations may be present that provide desired characteristics. Full length IgG should not have deletions of substantial portions of any of the regions. However, IgG molecules wherein one or several amino acid residues are deleted, without essentially altering the binding characteristics of the resulting IgG molecule, are embraced within the term “full length IgG”. For instance, such IgG molecules can have a deletion of between 1 and 10 amino acid residues, preferably in non-CDR regions, wherein the deleted amino acids are not essential for the antigen binding specificity of the IgG.

“Percent (%) identity” as referring to nucleic acid or amino acid sequences herein is defined as the percentage of residues in a candidate sequence that are identical with the residues in a selected sequence, after aligning the sequences for optimal comparison purposes. The percent sequence identity comparing nucleic acid sequences is determined using the AlignX application of the Vector NTI Advance® 11.5.2 software using the default settings, which employ a modified ClustalW algorithm (Thompson, J. D., Higgins, D. G., and Gibson T. J., (1994) Nuc. Acid Res. 22 (22): 4673-4680), the swgapdnamt score matrix, a gap opening penalty of 15 and a gap extension penalty of 6.66. Amino acid sequences are aligned with the AlignX application of the Vector NTI Advance® 11.5.2 software using default settings, which employ a modified ClustalW algorithm (Thompson, J. D., Higgins, D. G., and Gibson T. J., (1994) Nuc. Acid Res. 22 (22): 4673-4680), the blosum62mt2 score matrix, a gap opening penalty of 10 and a gap extension penalty of 0.1.

As an antibody typically recognizes an epitope of an antigen, and such an epitope may be present in other compounds as well, antibodies according to the present invention that “specifically recognize” an antigen, for example, EGFR or LGR5, may recognize other compounds as well, if such other compounds contain the same kind of epitope. Hence, the terms “specifically recognizes” with respect to an antigen and antibody interaction does not exclude binding of the antibodies to other compounds that contain the same kind of epitope.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein (so-called linear and conformational epitopes). Epitopes formed from contiguous, linear amino acids are typically retained on exposure to denaturing solvents, whereas epitopes formed by tertiary folding, conformation are typically lost on treatment with denaturing solvents. An epitope may typically include 3, 4, 5, 6, 7, 8, 9, 10, 11, 12, 13, 14 or 15 amino acids in a unique spatial conformation. Methods of determining spatial conformation of epitopes are known to persons of ordinary skill in the art and include techniques in the art for example, x-ray crystallography, HDX-MS and 2-dimensional nuclear magnetic resonance, pepscan, and alanine scan depending on the nature of the epitope (see, e.g., Epitope Mapping Protocols in Methods in Molecular Biology, Vol. 66, G. E. Morris, Ed. (1996)).

As used herein, the terms “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer).

The terms “treat,” “treating,” and “treatment,” as used herein, refer to any type of intervention or process performed on, or administering an active agent or combination of active agents to the subject with the objective of reversing, alleviating, ameliorating, inhibiting, or slowing down or preventing the progression, development, severity or recurrence of a symptom, complication, condition or biochemical indicia associated with a disease.

As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease in tumor size, decrease the presence of circulating tumor cells, reduce or prevent metastases of a tumor, slow or arrest tumor growth and/or prevent or delay tumor recurrence or relapse.

The term “effective amount” or “therapeutically effective amount” refer to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In some embodiments, an effective amount is an amount sufficient to delay tumor development. In some embodiments, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of an EGFR/LGR5 antibody and a topoisomerase I inhibitor, in combination, to effect a decrease in a cancer (for example a decrease in the number of cancer cells); slowing of progression of a cancer, or prevent regrowth or recurrence of the cancer, wherein the cancer is gastrointestinal cancer, preferably colorectal cancer.

The invention further provides a method for inhibiting growth of a cell that expresses EGFR and that expresses LGR5 in a system permissive for growth of the cell, the method comprising providing the system with an antibody and a topoisomerase I inhibitor as described herein. The system is preferably a culture system. The method preferably comprises culturing said cell in said system. The inhibition is preferably a decrease of at least 10% in cell number, tumor volume or tumor size, when compared to the number of cells or tumor volume/size resulting under otherwise similar conditions but for the presence of the antibody and/or topoisomerase I inhibitor of the invention. The inhibition is preferably a decrease of at least 20%, 30%, 40%, 50%, 60%, 70%, 80% or 90% in the number of cells, tumor volume or tumor size and/or increased progression free survival. The inhibition may also be a decrease of at least 10% in other parameters associated with tumour malignancy or dysplasia, such as the number of lumens per organoid, when compared to the number of lumens resulting under otherwise similar conditions but for the presence of the antibody and/or topoisomerase I inhibitor of the invention. The inhibition is preferably a decrease of at least 20%, 30%, 40%, 50%, 60%, 70%, 80% or 90% in the number of lumens per organoid and/or increased progression free survival.

For the avoidance of doubt the reference to growth of cells as used herein refers to a change in the number of cells. Inhibition of growth refers to a reduction in the number of cells that would otherwise have been obtained under otherwise similar conditions but for the presence of the antibody and/or topoisomerase I inhibitor of the invention. Increase in growth refers to an increase in the number of cells that would otherwise have been obtained. The growth of a cell typically refers to the proliferation of the cell. The reduction is compared to the growth/proliferation of the same cell under otherwise identical conditions in the absence of the antibody and/or topoisomerase I inhibitor of the invention.

The invention also provides a method for the treatment of an individual that has a gastrointestinal cancer or is at risk of having said cancer, the method comprising administering an antibody of the invention to the individual in need thereof. The individual is preferably an individual that has cancer. The cancer is preferably a gastrointestinal cancer. In a preferred embodiment the cancer is colorectal cancer.

The invention also provides a method for the prevention of metastasis or tumor recurrence to occur in an individual that has cancer, preferably a gastrointestinal cancer or is at risk of having said cancer, the method comprising administering an antibody or functional part, derivative and/or analogue thereof that comprises a variable domain that binds an extracellular part of EGFR and a variable domain that binds an extracellular part of LGR5 and a topoisomerase I inhibitor to the individual in need thereof. The individual is preferably an individual that has cancer or in case of tumor recurrence, an individual with radiographic diagnosis of return, or signs and symptoms of return of cancer after a period of improvement or response. The cancer is preferably a gastrointestinal cancer. In a preferred embodiment the cancer is colorectal cancer.

Prevention of metastasis is, in a preferred embodiment, prevention of metastasis from gastrointestinal cancer to non-gastrointestinal cancer such as metastasis in lung or liver tissue.

An effective amount of the combination therapy is administered according to the methods described herein in an “effective regimen” which refers to a combination of an EGFR/LGR5 antibody and a topoisomerase I inhibitor, wherein the order of administration, amount dosed and dosage frequency is adequate to effect treatment.

As stated above, cancer types, like CRC, can be related to the presence of oncogenic mutations, like those present in genes encoding phosphatidylinositol-4,5-bisphosphate 3-kinase catalytic subunit alpha (PIK3CA) or Kirsten RAt Sarcoma (KRAS). Mutations in both PIK3CA and KRAS are widely implicated in cancer-types like colorectal cancer. Prevalence of KRAS and PIK3C mutations in metastatic CRC is between 20 and 50% for different ethnic populations and up to 14.3%, respectively, while the PIK3CA C420R mutation has been detected in at least nine different types of cancer, including breast carcinoma, colorectal adenocarcinoma, esophageal carcinoma, brain lower grade glioma, lung squamous cell carcinoma, uterine corpus neoplasm, prostate adenocarcinoma, stomach adenocarcinoma and ovarian neoplasm (Prevalence of KRAS, BRAF, PI3K and EGFR mutations among Asian patients with metastatic colorectal cancer, Phua et al., Oncology Letters, 10:2519-2526 2014; the AACR Project GENIE Consortium. AACR Project GENIE: powering precision medicine through an international consortium. Cancer Discovery. 2017; 7 (8):818-831. Dataset Version 4; www.cancer.gov/research/key-initiatives/ras/ras-central/blog/2017/pik3ca.pdf). By July 2019, the Catalogue Of Somatic Mutations In Cancer (COSMIC), hosted by the UK Sanger Institute, featured 18 different tissue types carrying mutation PIK3C C420R (cancer.sanger.ac.uk/cosmic, mutation ID COSM757). In mutation in PIK3CA C420R, cysteine has been replaced by arginine at amino acid residue position 420 of the protein and in KRAS G12D, glycine residue of position 12 (G) has been mutated into aspartic acid (D) of KRAS.

One advantage of the present invention is that subjects having a KRAS and/or a PIK3CA mutation that underwent the combination treatment of antibody that binds LGR5 and EGFR and a topoisomerase I inhibitor did not display a significant reduction of body weight throughout the administration. In particular, subjects having KRAS G12D and/or PIK3CA C420R mutations did not display statistically significant body weight reduction.

Thus, in a preferred embodiment, the present invention relates to a method of treatment of cancer in subjects having a mutation in the gene coding for KRAS, preferably a mutation leading to G12D and/or in the gene coding for PIK3CA, preferably a mutation leading to C420R.

The cancer that is treated in a method or product for use in the treatment as described herein is preferably a breast carcinoma, a colorectal adenocarcinoma, an esophageal carcinoma, a brain glioma, preferably a lower grade brain glioma, a lung squamous cell carcinoma, an uterine corpus neoplasm, a prostate adenocarcinoma, a stomach adenocarcinoma or an ovarian neoplasma. The cancer is preferably a colorectal cancer, a pulmonary cancer, a gastrointestional cancer or an ovarian cancer, preferably a colorectal cancer. The cancer preferably has a mutation in the gene encoding KRAS, the gene encoding PIK3CA or a combination thereof. The KRAS mutation is preferably a mutation leading to a G12D amino acid substitution. The PIK3CA mutation is preferably a mutation leading to a C420R amino acid substitution.

A further advantage of the present invention is that no apparent signs of toxicity of the combination treatment of an antibody that binds LGR5 and EGFR and a topoisomerase I inhibitor were recorded in subjects having a KRAS and/or a PIK3CA mutation, in particular in subjects with KRAS G12D and/or PIK3CA C420R mutations.

As used herein, the terms “synergy”, “therapeutic synergy”, and “synergistic effect” refer to a phenomenon where treatment of patients with a combination of therapeutic agents (e.g., an EGFR/LGR5 antibody in combination with a topoisomerase I inhibitor) manifests a therapeutically superior outcome to the outcome achieved by each individual constituent of the combination when used alone (see, e.g., T. H. Corbett et al., 1982, Cancer Treatment Reports, 66, 1187). In this context a therapeutically superior outcome includes one or more of the following (a) an increase in therapeutic response that is greater than either or both of the separate effects of each agent alone at the same dose as in the combination; (b) a decrease in the dose of one or more agents in the combination without a decrease in therapeutic efficacy; (c) a decrease in the incidence of adverse events while receiving a therapeutic benefit that is equal to or greater than the monotherapy of each agent at the same dose as in the combination, (d) a reduction in dose-limiting toxicities while receiving a therapeutic benefit that is greater than the monotherapy of each agent; (e) a delay or minimization of the induction of drug resistance. In xenograft models, a combination, used at its maximum tolerated dose, in which each of the constituents will be present at a dose generally not exceeding its individual maximum tolerated dose, manifests therapeutic synergy when decrease in tumor growth achieved by administration of the combination is greater than the value of the decrease in tumor growth of the best constituent when the constituent is administered alone. Synergism of a drug combination may be determined, for example, according to the combination index (CI) theorem of Chou-Talalay (Chou et al., Adv. Enzyme Regul. 1984; 22:27-55; Chou, Cancer Res. 2010; 70 (2):440-446).

“Relapse” or “recurrence” or “resurgence” are used interchangeably herein, and refer to the radiographic diagnosis of return, or signs and symptoms of return of cancer after a period of improvement or response.

Topoisomerase inhibitors are chemical compounds that block the action of a topoisomerase (topoisomerase I and II). Topoisomerases are a type of enzyme that controls changes in DNA structure by catalyzing the breaking and rejoining of the phosphodiester backbone of DNA strands during the normal cell cycle.

Human topoisomerases have become targets for cancer chemotherapy treatments. Without being bound by theory it is thought that topoisomerase inhibitors generate single and double stranded breaks in the genome of a cell that affect the stability of the genome in the cells. Introduction of such breaks can result in apoptosis and cell death.

In the present invention the human topoisomerase inhibitor is preferably an inhibitor of human topoisomerase I. A non-limiting example of such a topoisomerase inhibitor is camptothecin (CPT). The CPT is long known to have anticancer properties. CPT has a relatively low solubility. Derivatives of CPT where found with a better activity. CPT derivatives/analogues have been approved and are used in cancer chemotherapy today. Examples of suitable topoisomerase I inhibitors in humans are camptothecin, topotecan, lamellarin D and irinotecan. In one embodiment a “topoisomerase I inhibitor”, as used herein, includes, but is not limited to, topotecan, irinotecan, gimatecan, camptothecin and its analogues, 9-nitrocamptot ecin and the macromolecular camptothecin conjugate PNU-166148 (compound A1 in WO 99/17804).

Irinotecan (CPT-11), a semisynthetic derivative of camptothecin, is a topoisomerase-I inhibitor which is active against a variety of solid tumors, including colorectal, pulmonary, gastric and ovarian cancer. Irinotecan is a prodrug, which is hydrolyzed by liver carboxylesterase to produce the active metabolite SN-38. SN-38 is eliminated by glucuronidation, which depends on hepatic UDP glucuronosyltransferase family 1, member A1 cluster (UGTA1) enzymes. Genotype UGT1A1*28 has been found to be associated with decreased SN-38 glucuronidation. Approximately 10% of North Americans carry 2 copies of the UGT1A1*28 allele (homozygous, UGT1A1*28/*28), and are more likely to develop neutropenia following irinotecan therapy (Dean L. Irinotecan Therapy and UGT1A1 Genotype. 2015 [Updated 2018 Apr. 4]. In: Pratt V, McLeod H, Rubinstein W, et al., editors. Medical Genetics Summaries [Internet]. Bethesda (Md.): National Center for Biotechnology Information (US); 2012; Available from: www.ncbi.nlm.nih.gov/books/NBK294473/). Subjects may be screened for the presence of one or more UGT1A1*28 alleles. Preferably, a subject being administered irinotecan does not carry one or more UGT1A1*28 alleles, more preferably said subject is not homozygous for allele UGT1A1*28.

Irinotecan and other human topoisomerase inhibitors have been used in the clinic for quite some time and appropriate dosage information is available to the person of ordinary skill in the art. For instance, irinotecan can be administered 70-350 mg/m² weekly, biweekly, every three weeks. Other regimes provide 120-150 mg/m² on day 1 and 8 ever three weeks. Yet further schedules include 125 mg/m² for 4 weeks followed by a two week interval. 50 mg/m² day 1-5 every three weeks and 20 mg/m² d1-5 of weeks 1, 2, 4 and 5. The administration expressed herein refers to the amount (in mg) per body surface area (in m²) of the subject per indicated time point.

An antibody or a functional part, derivative and/or analogue thereof as described herein comprises a variable domain that binds an extracellular part of the epidermal growth factor (EGF) receptor and a variable domain that binds LGR5. The EGFR is preferably a human EGFR. The LGR5 is preferably a human LGR5. The antibody or a functional part, derivative and/or analogue thereof as described herein comprises a variable domain that binds an extracellular part of a human epidermal growth factor (EGF) receptor and a variable domain that binds a human LGR5.

Epidermal growth factor (EGF) receptor (EGFR, ErbB1, or HERD is a member of a family of four receptor tyrosine kinases (RTKs), named Her- or cErbB-1, -2, -3 and -4. EGFR is known under various synonyms, the most common of which is EGFR. EGFR has an extracellular domain (ECD) that is composed of four sub-domains, two of which are involved in ligand binding and two of which are involved in homo-dimerisation and hetero-dimerisation. EGFR integrates extracellular signals from a variety of ligands to yield diverse intracellular responses. A major signal transduction pathway activated by EGFR is composed of the Ras-mitogen-activated protein kinase (MAPK) mitogenic signaling cascade. Activation of this pathway is initiated by the recruitment of Grb2 to tyrosine phosphorylated EGFR. This leads to activation of Ras through the Grb2-bound Ras-guanine nucleotide exchange factor Son of Sevenless (SOS). In addition, the PI3-kinase-Akt signal transduction pathway is also activated by EGFR, although this activation is much stronger in case there is co-expression of ErbB-3 (HER3). The EGFR is implicated in several human epithelial malignancies, notably cancers of the breast, bladder, non-small cell lung cancer lung, colon, ovarian head and neck and brain. Activating mutations in the gene have been found, as well as over-expression of the receptor and of its ligands, giving rise to autocrine activation loops. This RTK has therefore been extensively used as target for cancer therapy. Both small-molecule inhibitors targeting the RTK and monoclonal antibodies (mAbs) directed to the extracellular ligand-binding domains have been developed and have shown hitherto several clinical successes, albeit mostly for a select group of patients. The database accession number for the human EGFR protein and the gene encoding it is GenBank NM_005228.3. This accession number is primarily given to provide a further method of identification of EGFR protein as a target, the actual sequence of the EGFR protein bound by an antibody may vary, for instance because of a mutation in the encoding gene such as those occurring in some cancers or the like. The words cancer and tumor are used herein and typically both refer to cancer, unless otherwise specifically stated.

Where reference herein is made to EGFR, the reference refers to human EGFR unless otherwise stated. The variable domain antigen-binding site that binds EGFR, binds EGFR and a variety of variants thereof such as those expressed on some EGFR positive tumors.

The term “LGR” refers to the family of proteins known as Leucine-rich repeat-containing G-protein coupled receptors. Several members of the family are known to be involved in the WNT signaling pathway, of note LGR4; LGR5 and LGR6.

LGR5 is Leucine-Rich Repeat Containing G Protein-Coupled Receptor 5. Alternative names for the gene or protein are Leucine-Rich Repeat Containing G Protein-Coupled Receptor 5; Leucine-Rich Repeat-Containing G Protein-Coupled Receptor 5; G-Protein Coupled Receptor HG38; G-Protein Coupled Receptor 49; G-Protein Coupled Receptor 67; GPR67; GPR49; Orphan G Protein-Coupled Receptor HG38; G Protein-Coupled Receptor 49; GPR49; HG38 and FEX. A protein or antibody of the invention that binds LGR5, binds human LGR5. The LGR5 binding protein or antibody of the invention may, due to sequence and tertiary structure similarity between human and other mammalian orthologs, also bind such an ortholog but not necessarily so. Database accession numbers for the human LGR5 protein and the gene encoding it are (NC_000012.12; NT_029419.13; NC_018923.2; NP_001264155.1; NP_001264156.1; NP_003658.1). The accession numbers are primarily given to provide a further method of identification of LGR5 as a target, the actual sequence of the LGR5 protein bound may vary, for instance because of a mutation in the encoding gene such as those occurring in some cancers or the like. The LGR5 antigen binding site binds LGR5 and a variety of variants thereof, such as those expressed by some LGR5 positive tumor cells.

In the context of the present invention a cell is said to express LGR5 if the cell comprises detectable RNA that codes for LGR5. Expression can often also be detected by incubating the cell with an antibody that binds to LGR5. However, some cells do not express the protein high enough for such an LGR5 antibody test. In such cases mRNA or other forms of nucleic acid sequence detection is preferred.

Where herein accession numbers or alternative names of proteins/genes are given, they are primarily given to provide a further method of identification of the mentioned protein as a target, the actual sequence of the target protein bound by an antibody of the invention may vary, for instance because of a mutation and/or alternative splicing in the encoding gene such as those occurring in some cancers or the like. The target protein is bound by the antibody as long as the epitope is present in the protein and the epitope is accessible to the antibody.

An antibody or a functional part, derivative and/or analogue thereof as described herein preferably interferes with the binding of a ligand for EGFR to EGFR. The term “interferes with binding” as used herein means that binding of the antibody or a functional part, derivative and/or analogue thereof to the EGFR competes with the ligand for binding to EGF receptor. The antibody or a functional part, derivative and/or analogue thereof may diminish ligand binding, displace ligand when this is already bound to the EGF receptor or it may, for instance through steric hindrance, at least partially prevent that ligand can bind to the EGF receptor.

An EGFR antibody of the invention preferably inhibits respectively EGFR ligand-induced signaling, measured as ligand-induced growth of BxPC3 cells (ATCC CRL-1687) or BxPC3-luc2 cells (Perkin Elmer 125058) or ligand-induced cell death of A431 cells (ATCC CRL-1555). The mentioned EGFR antibody can reduce ligand induced signaling by at least 5%, 10%, 15%, 20%, 25%, 30%, 35%, preferably 40%, 45%, 50%, 55%, 60%, more preferably 70%, 80%, 85%, and most preferably 90%, 95%, 99%, or 100% compared to the ligand induced effect in the presence of a neutral substance or negative control as measured in an assay known in the art. EGFR can bind a number of ligands and stimulate growth of the mentioned BxPC3 cells or BxPC3-luc2 cells. In the presence of an EGFR ligand the growth of BxPC3 or BxPC3-luc2 cells is stimulated. EGFR ligand-induced growth of BxPC3 cells can be measured by comparing the growth of the cells in the absence and presence of the ligand. The preferred EGFR ligand for measuring EGFR ligand-induced growth of BxPC3 or BxPC3-luc2 cells is EGF. The ligand-induced growth is preferably measured using saturating amounts of ligand. In a preferred embodiment EGF is used in an amount of 100 ng/ml of culture medium. EGF is preferably the EGF R&D systems, cat. nr. 396-HB and 236-EG (see also WO2017/069628; which is incorporated by reference herein).

An EGFR antibody of the invention preferably inhibits EGFR ligand induced growth of BxPC3 cells (ATCC CRL-1687) or BxPC3-luc2 cells (Perkin Elmer 125058). The mentioned EGFR antibody can reduce ligand induced growth signaling by at least 5%, 10%, 15%, 20%, 25%, 30%, 35%, preferably 40%, 45%, 50%, 55%, 60%, more preferably 70%, 80%, 85%, and most preferably 90%, 95%, 99%, or 100% compared to the ligand induced growth induced by a neutral substance or negative control as measured in an assay known in the art. EGFR can bind a number of ligands and stimulate growth of the mentioned BxPC3 cells or BxPC3-luc2 cells. In the presence of a ligand the growth of BxPC3 or BxPC3-luc2 cells is stimulated. EGFR ligand-induced growth of BxPC3 cells can be measured by comparing the growth of the cells in the absence and presence of the ligand. The preferred EGFR ligand for measuring EGFR ligand-induced growth of BxPC3 or BxPC3-luc2 cells is EGF. The ligand-induced growth is preferably measured using saturating amounts of ligand. In a preferred embodiment EGF is used in an amount of 100 ng/ml of culture medium. EGF is preferably the EGF of R&D systems, cat. nr. 396-HB and 236-EG (see also WO2017/069628; which is incorporated by reference herein).

Whether an antibody of the invention inhibits signaling or inhibits growth in a multispecific format is preferably determined by the method as described herein above using a monospecific monovalent or monospecific bivalent version of the antibody. Such an antibody preferably has binding sites for the receptor of which signaling is to be determined. A monospecific monovalent antibody can have a variable domain with an irrelevant binding specificity such as a tetanus toxoid specificity. A preferred antibody is a bivalent monospecific antibody wherein the antigen binding variable domains consist of variable domains that bind the EGF-receptor family member.

An antibody or a functional part, derivative and/or analogue thereof as described herein comprises a variable domain that binds an extracellular part of LGR5.

The variable domain that binds an extracellular part of LGR5 preferably binds an epitope that is located within amino acid residues 21-118 of the sequence of FIG. 1 of which amino acid residues D43; G44, M46, F67, R90, and F91 are involved in binding of the antibody to the epitope.

The LGR5 variable domain is preferably a variable domain wherein one or more of the amino acid residue substitutions in LGR5 of D43A; G44A, M46A, F67A, R90A, and F91A reduces the binding of the variable domain to LGR5.

The epitope on an extracellular part of LGR5 is preferably located within amino acid residues 21-118 of the sequence of FIG. 1. It is preferably an epitope wherein the binding of the LGR5 variable domain to LGR5 is reduced by one or more of the following amino acid residue substitutions D43A; G44A, M46A, F67A, R90A, and F91A in LGR5.

The invention further provides an antibody with a variable domain that binds an extracellular part of EGFR and a variable domain that binds an extracellular part of LGR5 wherein the LGR5 variable domain binds an epitope on LGR5 that is located within amino acid residues 21-118 of the sequence of FIG. 1

The epitope on LGR5 is preferably a conformational epitope. The epitope is preferably located within amino acid residues 40-95 of the sequence of FIG. 1. The binding of the antibody to LGR5 is preferably reduced with one or more of the following amino acid residue substitutions D43A; G44A, M46A, F67A, R90A, and F91A.

Without being bound by theory it is believed that M46, F67, R90, and F91 of LGR5 as depicted in FIG. 1, are contact residues for a variable domain as indicated herein above, i.e. the antigen-binding site of a variable domain that binds the LGR5 epitope. That amino acid residue substitution D43A and G44A reduces the binding of an antibody can be due to the fact that these are also contact residues, however, it is also possible that these amino acid residue substitution induce a (slight) modification of the conformation of the part of LGR5 that has one or more of the other contact residues (i.e. at positions 46, 67, 90 or 91) and that conformation change is such that antibody binding is reduced. The epitope is characterized by the mentioned amino acid substitutions. Whether an antibody binds the same epitope can be determined in various ways. In the examples a preferred method is described. The method utilizes CHO cells. The CHO cells express LGR5 on the cell membrane, or on alanine substitution mutant, preferably a mutant comprising one or more of the substitutions M46A, F67A, R90A, or F91A. A test antibody is contacted with the CHO cells and binding of the antibody to the cells compared. A test antibody binds the epitope if it binds to LGR5 and to a lesser extent to an LGR5 with a M46A, F67A, R90A, or F91A substitution. Comparing binding with a panel of mutants each comprising one alanine residue substitution is preferred. Such binding studies are well known in the art. Often the panel comprises single alanine substitution mutants covering essentially all amino acid residues. For LGR5 the panel only needs to cover the extracellular part of the protein and a part that warrants association with the cell membrane of course, when cell are used. Expression of a particular mutant can be compromised but this is easily detected by one or more LGR5 antibodies that bind to different region(s). If expression is also reduced for these control antibodies the level or folding of the protein on the membrane is compromised for this particular mutant. Binding characteristics of the test antibody to the panel readily identifies whether the test antibodies exhibits reduced binding to mutants with a M46A, F67A, R90A, or F91A substitution and thus whether the test antibody is an antibody of the invention. Reduced binding to mutants with a M46A, F67A, R90A, or F91A substitution also identifies the epitope to be located within amino acid residues 21-118 of the sequence of FIG. 1. In a preferred embodiment the panel includes a D43A substitution mutant; a G44A substitution mutant of both. The antibody with the VH sequence of the VH of MF5816 exhibits reduced binding to these substitution mutants.

Without being bound by any theory it is believed that amino acid residues 1462; G465; K489; 1491; N493; and C499 as depicted FIG. 2 are involved in binding an epitope by an antibody comprising a variable domain as indicated herein above. Involvement in binding is preferably determined by observing a reduced binding of the variable domain to an EGFR with one or more of the amino acid residue substitutions selected from I462A; G465A; K489A; I491A; N493A; and C499A.

In one aspect the variable domain that binds an epitope on an extracellular part of human EGFR is a variable domain that binds an epitope that is located within amino acid residues 420-480 of the sequence depicted in FIG. 2. Preferably the binding of the variable domain to EGFR is reduced by one or more of the following amino acid residue substitutions I462A; G465A; K489A; I491A; N493A; and C499A in EGFR. The binding of the antibody to human EGFR preferably interferes with the binding of EGF to the receptor. The epitope on EGFR is preferably a conformational epitope. In one aspect the epitope is located within amino acid residues 420-480 of the sequence depicted in FIG. 2, preferably within 430-480 of the sequence depicted in FIG. 2; preferably within 438-469 of the sequence depicted in FIG. 2.

Without being bound by theory it is believed that the contact residues of the epitope, i.e. where the variable domain contacts the human EGFR are likely 1462; K489; 1491; and N493. The amino acid residues G465 and C499 are likely indirectly involved in the binding of the antibody to EGFR, probably because mutation by substitution into an alanine induces a (slight) conformational alteration of the epitope resulting in a reduced binding to the epitope.

The variable domain that binds human EGFR, is preferably a variable domain with a heavy chain variable region that comprises at least the CDR3 sequence of the VH of MF3755 as depicted in FIG. 8 or a CDR3 sequence that differs in at most three, preferably in at most two, preferably in no more than one amino acid from a CDR3 sequence of the VH of MF3755 as depicted in FIG. 8.

The variable domain that binds human EGFR, is preferably a variable domain with a heavy chain variable region that comprises at least the CDR1, CDR2 and CDR3 sequences of the VH of MF3755 as depicted in FIG. 8; or the CDR1, CDR2 and CDR3 sequences of the VH of MF3755 as depicted in FIG. 8 with at most three, preferably at most two, preferably at most one amino acid substitutions.

The variable domain that binds human EGFR, is preferably a variable domain with a heavy chain variable region that comprises the sequence of the VH chain of MF3755 as depicted in FIG. 8; or the amino acid sequence of the VH chain of MF3755 depicted in FIG. 8 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain of MF3755.

In one embodiment the invention provides an antibody comprising a variable domain that binds an extracellular part of EGFR and a variable domain that binds an extracellular part of LGR5,

wherein a heavy chain variable region of said variable domain comprises at least the CDR3 sequence of an EGFR specific heavy chain variable region selected from the group consisting of MF3370; MF3755; MF4280 or MF4289 as depicted in FIG. 8 or wherein a heavy chain variable region of said variable domain comprises a heavy chain CDR3 sequence that differs in at most three, preferably in at most two, preferably in no more than one amino acid from a CDR3 sequence of a VH selected from the group consisting of MF3370; MF3755; MF4280 or MF4289 as depicted in FIG. 8. Said variable domain preferably comprises a heavy chain variable region comprising at least the CDR3 sequence of MF3370; MF3755; MF4280 or MF4289 as depicted in FIG. 8.

Said variable domain preferably comprises a heavy chain variable region comprising at least the CDR1, CDR2 and CDR3 sequences of an EGFR specific heavy chain variable region selected from the group consisting of MF3370; MF3755; MF4280 or MF4289 as depicted in FIG. 8, or heavy chain variable region comprising at least CDR1, CDR2 and CDR3 sequences that differ in at most three, preferably in at most two, preferably in at most one amino acid from the CDR1, CDR2 and CDR3 sequences of an EGFR specific heavy chain variable region selected from the group consisting of MF3370; MF3755; MF4280 or MF4289 as depicted in FIG. 8. Said variable domain preferably comprises a heavy chain variable region comprising at least the CDR1, CDR2 and CDR3 sequences of MF3370; MF3755; MF4280 or MF4289 as depicted in FIG. 8. A preferred heavy chain variable region is MF3755. Another preferred heavy chain variable region is MF4280.

The antibody comprising a variable domain that binds an extracellular part of EGFR and a variable domain that binds an extracellular part of LGR5, wherein the EGFR binding variable domains has a CDR3, a CDR1, CDR2, and CDR3 and/or a VH sequence as indicated herein above preferably has a variable domain that binds LGR5 that comprises at least the CDR3 sequence of an LGR5 specific heavy chain variable region selected from the group consisting of MF5790; MF5803; MF5805; MF5808; MF5809; MF5814; MF5816; MF5817; or MF5818 as depicted in FIG. 8 or a heavy chain CDR3 sequence that differs in at most three, preferably in at most two, preferably in no more than one amino acid from a CDR3 sequence of a VH selected from the group consisting of MF5790; MF5803; MF5805; MF5808; MF5809; MF5814; MF5816; MF5817; or MF5818 as depicted in FIG. 8. Said variable domain preferably comprises a heavy chain variable region comprising at least the CDR3 sequence of MF5790; MF5803; MF 5805; MF5808; MF5809; MF5814; MF5816; MF5817; or MF5818 as depicted in FIG. 8.

The LGR5 variable domain preferably comprises a heavy chain variable region comprising at least the CDR1, CDR2 and CDR3 sequences of an LGR5 specific heavy chain variable region selected from the group consisting of MF5790; MF5803; MF5805; MF5808; MF5809; MF5814; MF5816; MF5817; or MF5818 as depicted in FIG. 8, or heavy chain CDR1, CDR2 and CDR3 sequences that differ in at most three, preferably in at most two, preferably in at most one amino acid from the CDR1, CDR2 and CDR3 sequences of LGR5 specific heavy chain variable region selected from the group consisting of MF5790; MF5803; MF5805; MF5808; MF5809; MF5814; MF5816; MF5817; or MF5818 as depicted in FIG. 8. Said variable domain preferably comprises a heavy chain variable region comprising at least the CDR1, CDR2 and CDR3 sequences of MF5790; MF5803; MF5805; MF5808; MF5809; MF5814; MF5816; MF5817; or MF5818 as depicted in FIG. 8. Preferred heavy chain variable regions are MF5790; MF5803; MF5814; MF5816; MF5817; or MF5818. Particularly preferred heavy chain variable regions are MF5790; MF5814; MF5816; and MF5818; preferably MF5814, MF5818 and MF5816, heavy chain variable region MF5816 is particularly preferred. Another preferred heavy chain variable region is MF5818.

It has been shown that the antibodies comprising one or more variable domains with a heavy chain variable region MF3755 or one or more CDRs thereof have a better effectivity when used to inhibit growth of an EGFR ligand responsive cancer or cell. In the context of bispecific or multispecific antibodies, an arm of the antibody comprising a variable domain with a heavy chain variable region MF3755 or one or more CDRs thereof combines well with an arm comprising a variable domain with a heavy chain variable region MF5818 or one or more CDRs thereof.

VH chains of variable domains that bind EGFR or LGR5 can have one or more amino acid substitutions with respect to the sequence depicted in FIG. 8. A VH chain preferably has an amino acid sequence of an EGFR or LGR5 VH of FIG. 8, having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 8.

CDR sequences can have one or more an amino acid residue substitutions with respect to a CDR sequence in the figures. Such one or more substitutions are for instance made for optimization purposes, preferably in order to improve binding strength or the stability of the antibody. Optimization is for instance performed by mutagenesis procedures where after the stability and/or binding affinity of the resulting antibodies are preferably tested and an improved EGFR specific CDR sequence or LGR5 specific CDR sequence is preferably selected. A skilled person is well capable of generating antibody variants comprising at least one altered CDR sequence according to the invention. For instance, conservative amino acid substitution may be applied. Examples of conservative amino acid substitution include the substitution of one hydrophobic residue such as isoleucine, valine, leucine or methionine for another hydrophobic residue, and the substitution of one polar residue for another polar residue, such as the substitution of arginine for lysine, glutamic acid for aspartic acid, or glutamine for asparagine.

Preferably, the mentioned at most 15, preferably 1, 2, 3, 4, 5, 6, 7, 8, 9 or 10 and preferably 1, 2, 3, 4 or 5 amino acid substitutions in a VH or VL as specified herein are preferably conservative amino acid substitutions. The amino acid insertions, deletions and substitutions in a VH or VL as specified herein are preferably not present in the CDR3 region. The mentioned amino acid insertions, deletions and substitutions are preferably also not present in the CDR1 and CDR2 regions. The mentioned amino acid insertions, deletions and substitutions are preferably also not present in the FR4 region.

The mentioned at most 15, preferably 1, 2, 3, 4, 5, 6, 7, 8, 9 or 10 and preferably 1, 2, 3, 4 or 5 amino acid substitutions are preferably conservative amino acid substitutions, the insertions, deletions, substitutions or a combination thereof are preferably not in the CDR3 region of the VH chain, preferably not in the CDR1, CDR2 or CDR3 region of the VH chain and preferably not in the FR4 region.

An antibody comprising a variable domain that binds an extracellular part of EGFR and a variable domain that binds an extracellular part of LGR5 preferably comprises
the amino acid sequence of VH chain MF3755 as depicted in FIG. 8; or
the amino acid sequence of VH chain MF3755 as depicted in FIG. 8 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
wherein the VH chain of the variable domain that binds LGR5 comprises
the amino acid sequence of VH chain MF5790 as depicted in FIG. 8; or
the amino acid sequence of VH chain MF5790 as depicted in FIG. 8 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

An antibody comprising a variable domain that binds an extracellular part of EGFR and a variable domain that binds an extracellular part of LGR5 preferably comprises
the amino acid sequence of VH chain MF3755 as depicted in FIG. 8; or
the amino acid sequence of VH chain MF3755 as depicted in FIG. 8 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
wherein the VH chain of the variable domain that binds LGR5 comprises
the amino acid sequence of VH chain MF5803 as depicted in FIG. 8; or
the amino acid sequence of VH chain MF5803 as depicted in FIG. 8 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

An antibody comprising a variable domain that binds an extracellular part of EGFR and a variable domain that binds an extracellular part of LGR5 preferably comprises
the amino acid sequence of VH chain MF3755 as depicted in FIG. 8; or
the amino acid sequence of VH chain MF3755 as depicted in FIG. 8 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
wherein the VH chain of the variable domain that binds LGR5 comprises
the amino acid sequence of VH chain MF5814 as depicted in FIG. 8; or
the amino acid sequence of VH chain MF5814 as depicted in FIG. 8 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

An antibody comprising a variable domain that binds an extracellular part of EGFR and a variable domain that binds an extracellular part of LGR5 preferably comprises
the amino acid sequence of VH chain MF3755 as depicted in FIG. 8; or
the amino acid sequence of VH chain MF3755 as depicted in FIG. 8 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
wherein the VH chain of the variable domain that binds LGR5 comprises
the amino acid sequence of VH chain MF5816 as depicted in FIG. 8; or
the amino acid sequence of VH chain MF5816 as depicted in FIG. 8 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

An antibody comprising a variable domain that binds an extracellular part of EGFR and a variable domain that binds an extracellular part of LGR5 preferably comprises
the amino acid sequence of VH chain MF3755 as depicted in FIG. 8; or
the amino acid sequence of VH chain MF3755 as depicted in FIG. 8 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
wherein the VH chain of the variable domain that binds LGR5 comprises
the amino acid sequence of VH chain MF5817 as depicted in FIG. 8; or
the amino acid sequence of VH chain MF5817 as depicted in FIG. 8 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

An antibody comprising a variable domain that binds an extracellular part of EGFR and a variable domain that binds an extracellular part of LGR5 preferably comprises
the amino acid sequence of VH chain MF3755 as depicted in FIG. 8 or
the amino acid sequence of VH chain MF3755 as depicted in FIG. 8 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
wherein the VH chain of the variable domain that binds LGR5 comprises
the amino acid sequence of VH chain MF5818 as depicted in FIG. 8; or
the amino acid sequence of VH chain MF5818 as depicted in FIG. 8 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

Additional variants of the disclosed amino acid sequences which retain EGFR or LGR5 binding can be obtained, for example, from phage display libraries which contain the rearranged human IGKV1-39/IGKJ1VL region (De Kruif et al. Biotechnol Bioeng. 2010 (106) 741-50), and a collection of VH regions incorporating amino acid substitutions into the amino acid sequence of an EGFR or LGR5 VH region disclosed herein, as previously described (e.g., WO2017/069628). Phages encoding Fab regions which bind EGFR or LGR5 may be selected and analyzed by flow cytometry, and sequenced to identify variants with amino acid substitutions, insertions, deletions or additions which retain antigen binding.

The light chain variable regions of the VH/VL EGFR and LGR5 variable domains of the EGFR/LGR5 antibody may be the same or different.

In some embodiments, the VL region of the VH/VL EGFR variable domain of the EGFR/LGR5 antibody is similar to the VL region of the VH/VL LGR5 variable domain. In certain embodiments, VL regions in the first and second VH/VL variable domains are identical.

In certain embodiments, the light chain variable region of one or both VH/VL variable domains of the EGFR/LGR5 antibody comprises a common light chain variable region. In some embodiments, the common light chain variable region of one or both VH/VL variable domains comprises a germline IgVκ1-39 variable region V-segment. In certain embodiment, the light chain variable region of one or both VH/VL variable domains comprises the kappa light chain V-segment IgVκ1-39*01. IgVκ1-39 is short for Immunoglobulin Variable Kappa 1-39 Gene. The gene is also known as Immunoglobulin Kappa Variable 1-39; IGKV139; IGKV1-39. External Ids for the gene are HGNC: 5740; Entrez Gene: 28930; Ensembl: ENSG00000242371. The amino acid sequence for a suitable V-region is provided in FIG. 9. The V-region can be combined with one of five J-regions. Preferred J-regions are jk1 and jk5, and the joined sequences are indicated as IGKV1-39/jk1 and IGKV1-39/jk5; alternative names are IgVκ1-39*01/IGJκ1*01 or IgVκ1-39*01/IGJκ5*01 (nomenclature according to the IMGT database worldwide web at imgt.org). In certain embodiments, the light chain variable region of one or both VH/VL variable domains comprises the kappa light chain IgVκ1-39*01/IGJκ1*01 or IgVκ1-39*01/IGJκ1*05 (described in FIG. 9).

In some embodiments, the light chain variable region of one or both VH/VL variable domains of the EGFR/LGR5 bispecific antibody comprises an LCDR1 comprising the amino acid sequence QSISSY (described in FIG. 9), an LCDR2 comprising the amino acid sequence AAS (described in FIG. 9), and an LCDR3 comprising the amino acid sequence QQSYSTP (described in FIG. 9) (i.e., the CDRs of IGKV1-39 according to IMGT). In some embodiments, the light chain variable region of one or both VH/VL variable domains of the EGFR/LGR5 antibody comprises an LCDR1 comprising the amino acid sequence QSISSY (described in FIG. 9), an LCDR2 comprising the amino acid sequence AASLQS (described in FIG. 9), and an LCDR3 comprising the amino acid sequence QQSYSTP (described in FIG. 9).

In some embodiments, one or both VH/VL variable domains of the EGFR/LGR5 antibody comprise a light chain variable region comprising an amino acid sequence that is at least 90%, preferably at least 95%, more preferably at least 97%, more preferably at least 98%, more preferably at least 99% identical or 100% identical to the amino acid sequence of set forth in FIG. 9. In some embodiments, one or both VH/VL variable domains of the EGFR/LGR5 antibody comprise a light chain variable region comprising an amino acid sequence that is at least 90%, preferably at least 95%, more preferably at least 97%, more preferably at least 98%, more preferably at least 99% identical or 100% identical to the amino acid sequence of set forth in FIG. 9.

For example, in some embodiments, the variable light chain of one or both VH/VL variable domains of the EGFR/LGR5 antibody can have from 0 to 10, preferably from 0 to 5 amino acid insertions, deletions, substitutions, additions or a combination thereof with respect to a sequence in FIG. 9. In some embodiments, the light chain variable region of one or both VH/VL variable domains of the EGFR/LGR5 antibody comprises from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

In other embodiments, the light chain variable region of one or both VH/VL variable domains of the EGFR/LGR5 antibody comprises the amino acid sequence of a sequence as depicted in FIG. 9. In certain embodiments, both VH/VL variable domains of the EGFR/LGR5 antibody comprise identical VL regions. In one embodiment, the VL of both VH/VL variable domains of the EGFR/LGR5 bispecific antibody comprises the amino acid sequence set forth in FIG. 9. In one embodiment, the VL of both VH/VL variable domains of the EGFR/LGR5 bispecific antibody comprises the amino acid sequence set forth in FIG. 9.

The EGFR/LGR5 antibody as described herein is preferably a bispecific antibody having two variable domains, one that binds EGFR and another that binds LGR5 as described herein.

EGFR/LGR5 bispecific antibodies for use in the methods disclosed herein can be provided in a number of formats. Many different formats of bispecific antibodies are known in the art, and have been reviewed by Kontermann (Drug Discov Today, 2015 July; 20 (7):838-47; MAbs, 2012 March-April:4 (2):182-97) and in Spiess et al., (Alternative molecular formats and therapeutic applications for bispecific antibodies. Mol. Immunol. (2015) http://dx.doi.org/10.1016/j.molimm.2015.01.003), which are each incorporated herein by reference. For example, bispecific antibody formats that are not classical antibodies with two VH/VL combinations, have at least a variable domain comprising a heavy chain variable region and a light chain variable region. This variable domain may be linked to a single chain Fv-fragment, monobody, a VH and a Fab-fragment that provides the second binding activity.

In some embodiments, the EGFR/LGR5 bispecific antibodies used in the methods provided herein are generally of the human IgG subclass (e.g., for instance IgG1, IgG2, IgG3, IgG4). In certain embodiments, the antibodies are of the human IgG1 subclass. Full length IgG antibodies are preferred because of their favorable half-life and for reasons of low immunogenicity. Accordingly, in certain embodiments, the EGFR/LGR5 bispecific antibody is a full length IgG molecule. In an embodiment, the EGFR/LGR5 bispecific antibody is a full length IgG1 molecule.

Accordingly, in certain embodiments, the EGFR/LGR5 bispecific antibody comprises a fragment crystallizable (Fc). The Fc of the EGFR/LGR5 bispecific antibody is preferably comprised of a human constant region. A constant region or Fc of the EGFR/LGR5 bispecific antibody may contain one or more, preferably not more than 10, preferably not more than 5 amino-acid differences with a constant region of a naturally occurring human antibody. For example, in certain embodiments, each Fab-arm of the bispecific antibodies may further include an Fc-region comprising modifications promoting the formation of the bispecific antibody, promoting stability and/or other features described herein.

Bispecific antibodies are typically produced by cells that express nucleic acid(s) encoding the antibody. Accordingly, in some embodiments, the bispecific EGFR/LGR5 antibodies disclosed herein are produced by providing a cell comprising one or more nucleic acids that encode the heavy and light chain variable regions and constant regions of the bispecific EGFR/LGR5 antibody. The cell is preferably an animal cell, more preferably a mammal cell, more preferably a primate cell, most preferably a human cell. A suitable cell is any cell capable of comprising and preferably of producing the EGFR/LGR5 bispecific antibody.

Suitable cells for antibody production are known in the art and include a hybridoma cell, a Chinese hamster ovary (CHO) cell, an NS0 cell or a PER-C6 cell. Various institutions and companies have developed cell lines for the large scale production of antibodies, for instance for clinical use. Non-limiting examples of such cell lines are CHO cells, NS0 cells or PER.C6 cells. In a particularly preferred embodiment said cell is a human cell. Preferably a cell that is transformed by an adenovirus E1 region or a functional equivalent thereof. A preferred example of such a cell line is the PER.C6 cell line or equivalent thereof. In a particularly preferred embodiment said cell is a CHO cell or a variant thereof. Preferably a variant that makes use of a Glutamine synthetase (GS) vector system for expression of an antibody. In one preferred embodiment, the cell is a CHO cell.

In some embodiments, the cell expresses the different light and heavy chains that make up the EGFR/LGR5 bispecific antibody. In certain embodiments, the cell expresses two different heavy chains and at least one light chain. In one preferred embodiment, the cell expresses a “common light chain” as described herein to reduce the number of different antibody species (combinations of different heavy and light chains). For example, the respective VH regions are cloned into expression vectors using methods known in the art for production of bispecific IgG (WO2013/157954; incorporated herein by reference), in conjunction with the rearranged human IGKV1 39/IGKJ1 (huVκ1 39) light chain. The huVκ1 39 was previously shown to be able to pair with more than one heavy chain thereby giving rise to antibodies with diverse specificities, which facilitates the generation of bispecific molecules (De Kruif et al. J. Mol. Biol. 2009 (387) 548 58; WO2009/157771).

An antibody producing cell that expresses a common light chain and equal amounts of the two heavy chains typically produces 50% bispecific antibody and 25% of each of the monospecific antibodies (i.e. having identical heavy light chain combinations). Several methods have been published to favor the production of the bispecific antibody over the production of the respective monospecific antibodies. Such is typically achieved by modifying the constant region of the heavy chains such that they favor heterodimerization (i.e. dimerization with the heavy chain of the other heavy/light chain combination) over homodimerization. In a preferred embodiment the bispecific antibody of the invention comprises two different immunoglobulin heavy chains with compatible heterodimerization domains. Various compatible heterodimerization domains have been described in the art. The compatible heterodimerization domains are preferably compatible immunoglobulin heavy chain CH3 heterodimerization domains. The art describes various ways in which such hetero-dimerization of heavy chains can be achieved.

One preferred method for producing the EGFR/LGR5 bispecific antibody is disclosed in U.S. Pat. Nos. 9,248,181 and 9,358,286. Specifically, preferred mutations to produce essentially only bispecific full length IgG molecules are the amino acid substitutions L351K and T366K (EU numbering) in the first CH3 domain (the ‘KK-variant’ heavy chain) and the amino acid substitutions L351D and L368E in the second domain (the ‘DE-variant’ heavy chain), or vice versa. As previously described, the DE-variant and KK-variant preferentially pair to form heterodimers (so-called ‘DEKK’ bispecific molecules). Homodimerization of DE-variant heavy chains (DEDE homodimers) or KK-variant heavy chains (KKKK homodimers) hardly occurs due to strong repulsion between the charged residues in the CH3-CH3 interface between identical heavy chains.

Accordingly, in one embodiment the heavy chain/light chain combination that comprises the variable domain that binds EGFR, comprises a DE variant of the heavy chain. In this embodiment the heavy chain/light chain combination that comprises the variable domain that binds LGR5 comprises a KK variant of the heavy chain.

A candidate EGFR/LGR5 IgG bispecific antibody can be tested for binding using any suitable assay. For example, binding to membrane-expressed EGFR or LGR5 on CHO cells can be assessed by flow cytometry (according to the FACS procedure as previously described in WO2017/069628). In one embodiment, the binding of a candidate EGFR/LGR5 bispecific antibody to LGR5 on CHO cells is demonstrated by flow cytometry, performed according to standard procedures known in the art. Binding to the CHO cells is compared with CHO cells that have not been transfected with expression cassettes for EGFR and/or LGR5. The binding of the candidate bispecific IgG1 to EGFR is determined using CHO cells transfected with an EGFR expression construct; a LGR5 monospecific antibody and an EGFR monospecific antibody, as well as an irrelevant IgG1 isotype control mAb are included in the assay as controls (e.g., an antibody which binds LGR5 and another antigen such as tetanus toxin (TT)).

The affinities of the LGR5 and EGFR Fabs of a candidate EGFR/LGR5 bispecific antibody for their targets can be measured by surface plasmon resonance (SPR) technology using a BIAcore T100. Briefly, an anti-human IgG mouse monoclonal antibody (Becton and Dickinson, cat. Nr. 555784) is coupled to the surfaces of a CM5 sensor chip using free amine chemistry (NHS/EDC). Then the bsAb is captured onto the sensor surface. Subsequently, the recombinant purified antigens human EGFR (Sino Biological Inc, cat. Nr. 11896-H07H) and human LGR5 protein are run over the sensor surface in a concentration range to measure on- and off-rates. After each cycle, the sensor surface is regenerated by a pulse of HCl and the bsAb is captured again. From the obtained sensorgrams, on- and off-rates and affinity values for binding to human LGR5 and EGFR are determined using the BIAevaluation software, as previously described for CD3 in US 2016/0368988.

An antibody of the invention is typically a bispecific full length antibody, preferably of the human IgG subclass. An antibody of the present invention is preferably of the human IgG1 subclass. Such antibodies of the invention have good ADCC properties which can, if desired, be enhanced by techniques known in the art, have favorable half-life upon in vivo administration to humans and CH3 engineering technology exists that can provide for modified heavy chains that preferentially form heterodimers over homodimers upon co-expression in clonal cells.

ADCC activity of an antibody can be improved when the antibody itself has a low ADCC activity, by slightly modifying the constant region of the antibody. Another way to improve ADCC activity of an antibody is by enzymatically interfering with the glycosylation pathway resulting in a reduced fucose. Several in vitro methods exist for determining the efficacy of antibodies or effector cells in eliciting ADCC. Among these are chromium-51 [Cr51] release assays, europium [Eu] release assays, and sulfur-35 [S35] release assays. Usually, a labeled target cell line expressing a certain surface-exposed antigen is incubated with antibody specific for that antigen. After washing, effector cells expressing Fc receptor CD16 are co-incubated with the antibody-labeled target cells. Target cell lysis is subsequently measured by release of intracellular label by a scintillation counter or spectrophotometry.

A bispecific antibody of the invention can in one embodiment be ADCC enhanced. A bispecific antibody of the invention can in one embodiment be afucosylated. A bispecific antibody of the invention preferably comprises a reduced amount of fucosylation of the N-linked carbohydrate structure in the Fc region, when compared to the same antibody produced in a normal CHO cell.

The antibody that comprises a variable domain that binds an extracellular part of EGFR and a variable domain that binds an extracellular part of LGR5 may further comprise one or more additional variable domains that can bind one or more further targets. The further target is preferably a protein, preferably a membrane protein comprising an extracellular part. Antibodies with more than two variable domains are known in the art. For instance, it is possible to attach an additional variable domain to a constant part of the antibody. An antibody with three or more variable domains is preferably a multivalent multimer antibody as described in PCT/NL2019/050199 which is incorporated by reference herein.

In one embodiment the antibody is a bispecific antibody comprising two variable domains, wherein one variable domain binds an extracellular part of EGFR and another variable domain binds an extracellular part of LGR5. The variable domains are preferably variable domains as described herein.

For the avoidance of doubt the reference to the growth of a cell as used herein refers to a change in the number of cells. Inhibition of growth refers to a reduction in the number of cells that would otherwise have been obtained. Increase in growth refers to an increase in the number of cells that would otherwise have been obtained. The growth of a cell typically refers to the proliferation of the cell.

A membrane protein as used herein is a cell membrane protein, such as a protein that is in the outer membrane of a cell, the membrane that separates the cell from the outside world. The membrane protein has an extracellular part. A membrane protein is at least on a cell if it contains a transmembrane region that is in the cell membrane of the cell.

Also provided is a pharmaceutical composition comprising an EGFR/LGR5 bispecific antibody, a topoisomerase I inhibitor and a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans, and includes any and all solvents, salts, dispersion media, coatings, antibacterial and antifungal agents, isotonic and absorption delaying agents, and the like that are physiologically compatible. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions. Liquid compositions for parenteral administration can be formulated for administration by injection or continuous infusion. Routes of administration by injection or infusion include intravesical, intratumoral, intravenous, intraperitoneal, intramuscular, intrathecal and subcutaneous. Depending on the route of administration (e.g., intravenously, subcutaneously, intra articularly and the like) the active compound may be coated in a material to protect the compound from the action of acids and other natural conditions that may inactivate the compound.

Pharmaceutical compositions suitable for administration to human patients are typically formulated for parenteral administration, e.g., in a liquid carrier, or suitable for reconstitution into liquid solution or suspension for intravenous administration. The compositions may be formulated in dosage unit form for ease of administration and uniformity of dosage.

Also included are solid preparations which are intended for conversion, shortly before use, to liquid preparations for either oral or parenteral administration. Such liquid forms include solutions, suspensions and emulsions.

The compositions and methods provided herein are particularly useful for the treatment of cancer in a patient, particularly gastrointestinal cancer. Accordingly, the compositions and methods may be used in the treatment of various malignancies.

As used herein, combined administration (co-administration) includes simultaneous administration of the EGFR/LGR5 bispecific antibody and topoisomerase I inhibitor in the same or different dosage form, separate administration or sequential administration. Accordingly, in some embodiments, an EGFR/LGR5 bispecific antibody may be used in a method for treating cancer in a subject, wherein the EGFR/LGR5 bispecific antibody is administered simultaneously, separately or sequentially with an topoisomerase I inhibitor. In other embodiments, an EGFR/LGR5 bispecific antibody may be used in the treatment of a cancer in a subject, wherein the EGFR/LGR5 bispecific antibody may be administered simultaneously, separately or sequentially with a topoisomerase I inhibitor.

In other embodiments, an EGFR/LGR5 bispecific antibody may be for use in the manufacture of a medicament for the treatment of cancer in a subject, wherein the EGFR/LGR5 bispecific antibody is administered simultaneously, separately or sequentially with a topoisomerase I inhibitor. In other embodiments, an EGFR/LGR5 bispecific antibody may be for use in the manufacture of a medicament for treating a cancer in a subject, wherein the EGFR/LGR5 bispecific antibody may be administered simultaneously, separately or sequentially with a topoisomerase I inhibitor. A product comprising an EGFR/LGR5 bispecific antibody and a topoisomerase I inhibitor may be a combined preparation for simultaneous, separate or sequential use in treating a cancer in a subject.

The EGFR/LGR5 bispecific antibody and topoisomerase I inhibitor can be administered according to a suitable dosage, route (e.g., intravenous, intraperitoneal, intramuscular, intrathecal or subcutaneous).

The EGFR/LGR5 bispecific antibody and topoisomerase I inhibitor can also be administered according to any suitable schedule. For example, the EGFR/LGR5 bispecific antibody and topoisomerase I inhibitor can be simultaneously administered in a single formulation. Alternatively, the EGFR/LGR5 bispecific antibody and topoisomerase I inhibitor can be formulated for separate administration, wherein they are administered concurrently or sequentially.

For example, in some embodiments, the EGFR/LGR5 bispecific antibody can be administered first followed by the administration of the topoisomerase I inhibitor, or vice versa. Dosage regimens in the above methods of treatment and uses are adjusted to provide the optimum desired response (e.g., a therapeutic response).

For example, a single bolus may be administered, several divided doses may be administered over time or the dose may be proportionally reduced or increased as indicated by the exigencies of the therapeutic situation. In one embodiment, the EGFR/LGR5 bispecific antibody is administered prior to administration of the topoisomerase I inhibitor, e.g., the EGFR/LGR5 bispecific antibody is administered into the patient first, followed from later by an administration of the topoisomerase I inhibitor. In one embodiment, the topoisomerase I inhibitor is administered prior to administration of the EGFR/LGR5 bispecific antibody, e.g., the topoisomerase I inhibitor is administered into the patient first, followed from later by administration of the EGFR/LGR5 bispecific antibody (e.g., one or more minutes, hours or days after). Such concurrent or sequential administration results in both the EGFR/LGR5 bispecific antibody and topoisomerase I inhibitor being simultaneously present in treated patients. Concurrent presence of both the EGFR/LGR5 bispecific antibody and topoisomerase I inhibitor will support EGFR/LGR5 bispecific antibody induced cancer treatment and EGFR/LGR5 bispecific antibody mediated inhibition of EGFR/LGR5 signaling.

In another embodiment, the topoisomerase I inhibitor and the EGFR/LGR5 bispecific antibody are administered simultaneously.

In one embodiment, a subject is administered a single dose of a topoisomerase I inhibitor and a single dose of the EGFR/LGR5 bispecific antibody. In some embodiments, the EGFR/LGR5 bispecific antibody and topoisomerase I inhibitor will be administered repeatedly, over a course of treatment. For example, in certain embodiments, multiple (e.g., 2, 3, 4, 5, 6, 7, 8, 9, 10 or more) doses of a topoisomerase I inhibitor and multiple (e.g., 2, 3, 4, 5, 6, 7, 8, 9, 10 or more) doses of an EGFR/LGR5 bispecific antibody are administered to a subject in need of treatment.

In some embodiments, administrations of a topoisomerase I inhibitor and an EGFR/LGR5 bispecific antibody may be done weekly, biweekly or monthly, in which regimen, they may be administered on the same day (e.g., simultaneously), or one after the other (e.g., one or more minutes, hours or days before or after one another). When administered separately, the EGFR/LGR5 bispecific antibody and topoisomerase I inhibitor may be, but are not necessarily administered according to the same administration (i.e., dosing) protocol. For example, one cycle of treatment may comprise administering the EGFR/LGR5 bispecific antibody one or multiple times, while a therapeutically effective dose of the topoisomerase I inhibitor may be administered either more or less frequently the EGFR/LGR5 bispecific antibody. In certain embodiments, administration of each dose of the topoisomerase I inhibitor and the EGFR/LGR5 bispecific antibody may be on the same day, or alternatively, the topoisomerase I inhibitor may be administered 1 or more days before or after the EGFR/LGR5 antibody.

In some embodiment, the dose of the EGFR/LGR5 bispecific antibody and/or topoisomerase I inhibitor is varied over time. For example, the EGFR/LGR5 bispecific antibody and/or topoisomerase I inhibitor may be initially administered at a high dose and may be lowered over time. In another embodiment, the EGFR/LGR5 bispecific antibody and/or topoisomerase I inhibitor is initially administered at a low dose and increased over time.

In another embodiment, the amount of the EGFR/LGR5 bispecific antibody and/or topoisomerase I inhibitor are administered is constant for each dose. In another embodiment, the amount of the EGFR/LGR5 bispecific antibody and/or topoisomerase I inhibitor varies with each dose. For example, the maintenance (or follow-on) dose of each can be higher or the same as the loading dose which is first administered. In another embodiment, the maintenance dose of the each can be lower or the same as the loading dose. A clinician may utilize preferred dosages as warranted by the condition of the patient being treated. The dose may depend upon a number of factors, including stage of disease, etc. The specific dose that should be administered based upon the presence of one or more of such factors is within the skill of the artisan. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In certain embodiments, the EGFR/LGR5 bispecific antibody is administered at a dose of 0.1, 0.3, 1, 2, 3, 4, 5, 6, 7, 8, 9, or 10 mg/kg body weight. In another embodiment, the EGFR/LGR5 bispecific antibody is administered at a dose of 0.5, 1, 2, 3, 4, 5, 6, 7, 8, 9, or 10 mg/kg body weight.

The treatment method described herein is typically continued for as long as the clinician overseeing the patient's care deems the treatment method to be effective, i.e., that the patient is responding to treatment. Non-limiting parameters that indicate the treatment method is effective may include one or more of the following: decrease in tumor cells; inhibition of tumor cell proliferation; tumor cell elimination; progression-free survival; appropriate response by a suitable tumor marker (if applicable).

With regard to the frequency of administering the EGFR/LGR5 bispecific antibody, one of ordinary skill in the art will be able to determine an appropriate frequency. For example, a clinician can decide to administer the EGFR/LGR5 bispecific antibody relatively infrequently (e.g., once every two weeks) and progressively shorten the period between doses as tolerated by the patient. With regard to frequency of administering the topoisomerase I inhibitor, the frequency for these agents can be determined in a similar fashion. Exemplary lengths of time associated with the course of therapy in accordance with the claimed method include: about one week; two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years; about five years; perpetual (e.g., ongoing maintenance therapy). The foregoing duration may be associated with one or multiple rounds/cycles of treatment.

The efficacy of the treatment methods provided herein can be assessed using any suitable means. In one embodiment, the clinical efficacy of the combination treatment is analyzed using cancer cell number reduction as an objective response criterion. Patients, e.g., humans, treated according to the methods disclosed herein preferably experience improvement in at least one sign of cancer. In some embodiments, one or more of the following can occur: the number of cancer cells can be reduced; cancer recurrence is prevented or delayed; one or more of the symptoms associated with cancer can be relieved to some extent. In addition, in vitro assays to determine the T cell mediated target cell lysis.

In another embodiment, the methods of treatment produce a comparable clinical benefit rate (CBR=CR (complete response), PR (partial response) or SD (stable disease)≥6 months) better than that achieved by an EGFR/LGR5 bispecific antibody or a topoisomerase I inhibitor (e.g., irinotecan) alone.

In some embodiment, the tumor cells are no longer detectable following treatment as described herein. In some embodiments, a subject is in partial or full remission. In certain embodiments, a subject has an increased overall survival, median survival rate, and/or progression free survival.

The combinations of the present invention (e.g., EGFR/LGR5 bispecific antibody in combination with topoisomerase I inhibitor) may also be used in conjunction with other well-known therapies that are selected for their particular usefulness against the cancer that is being treated. Combinations of the instant invention may alternatively be used sequentially with known pharmaceutically acceptable agent(s) when appropriate.

Methods for the safe and effective administration of chemotherapeutic agents are known to those skilled in the art. In addition, their administration is described in the standard literature. For example, the administration of many of the chemotherapeutic agents is described in the Physicians' Desk Reference (PDR), e.g., 1996 edition (Medical Economics Company, Montvale, N.J. 07645-1742, USA); the disclosure of which is incorporated herein by reference thereto.

It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

Also provided herein is a kit or a product which includes a pharmaceutical composition containing an EGFR/LGR5 bispecific antibody and a topoisomerase I inhibitor, and a pharmaceutically-acceptable carrier, in a therapeutically effective amount adapted for use in the preceding methods. In some embodiments, the kit or product optionally also can include instructions, e.g., comprising administration schedules, to allow a practitioner (e.g., a physician, nurse, or patient) to administer the composition contained therein to a patient having cancer.

In some embodiments, the kit or product include multiple packages of the single-dose pharmaceutical compositions each containing an effective amount of an EGFR/LGR5 bispecific antibody and a topoisomerase I inhibitor for a single administration in accordance with the methods provided above. Instruments or devices necessary for administering the pharmaceutical composition(s) also may be included in the kit or product. For instance, a kit or product may provide one or more pre-filled syringes containing a unit dosage of an EGFR/LGR5 bispecific antibody and a topoisomerase I inhibitor in the same container, or in separate containers to be administered as separate and distinct compositions.

In certain embodiments, one or both of an EGFR/LGR5 bispecific antibody and a topoisomerase I inhibitor is provided in a solid form suitable for reconstitution and subsequent administration in accordance with the accompanying instructions.

A functional part of an antibody as described herein comprises at least a variable domain that binds an extracellular part of EGFR and a variable domain that binds an extracellular part of LGR5 as described herein. It thus comprises the antigen binding parts of an antibody as described herein and typically contains the variable domains of the antibody. A variable domain of a functional part can be a single chain Fv-fragment or a so-called single domain antibody fragment. A single-domain antibody fragment (sdAb) is an antibody fragment with a single monomeric variable antibody domain. Like a whole antibody, it is able to bind selectively to a specific antigen. With a molecular weight of only 12-15 kDa, single-domain antibody fragments are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). Single-domain antibodies by themselves are not much smaller than normal antibodies (being typically 90-100kDa). Single-domain antibody fragments are mostly engineered from heavy-chain antibodies found in camelids; these are called VHH fragments (Nanobodies®). Some fishes also have heavy-chain only antibodies (IgNAR, ‘immunoglobulin new antigen receptor’), from which single-domain antibody fragments called VNAR fragments can be obtained. An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes. Non-limiting examples of such variable domains of antibody parts are VHH, Human Domain Antibodies (dAbs) and Unibodies. Preferred antibody parts or derivatives have at least two variable domains of an antibody or equivalents thereof. Non-limiting examples of such variable domains or equivalents thereof are F(ab)-fragments and Single chain Fv fragments. A functional part of a bispecific antibody comprises the antigen binding parts of the bispecific antibody, or a derivative and/or analogue of the binding parts. As mentioned herein above, the binding part of an antibody is encompassed in the variable domain.

In yet further embodiments, the composition or combination or kit or product includes one or more additional active agents.

All documents and references, including Genbank entries, patents and published patent applications, and websites, described herein are each expressly incorporated herein by reference to the same extent as if were written in this document in full or in part.

For the purpose of clarity and a concise description features are described herein as part of the same or separate embodiments, however, it will be appreciated that the scope of the invention may include embodiments having combinations of all or some of the features described.

The invention is now described by reference to the following examples, which are illustrative only, and are not intended to limit the present invention. While the invention has been described in detail and with reference to specific embodiments thereof, it will be apparent to one of skill in the art that various changes and modifications can be made thereto without departing from the spirit and scope thereof.

### BRIEF DESCRIPTION OF THE DRAWINGS

FIG. 1 Human LGR5 sequence.

FIG. 2 Human EGFR sequence.

FIG. 3 Effect of treatments on tumor volume in M005 orthotopic PDX model of CRC. (a) Injection frequency, dosing and sites of injections during the treatment period; (b) Fold change in tumor volume over time; and (c) Dot plot showing fold change per mouse at 6 weeks.

FIG. 4 (a) Tumor volume before and after treatment release in mouse model M005. Treatment was stopped after 9 weeks and tumor volume in the same mice monitored for a further 3 weeks. Numbers beneath each group indicate reasons why not all mice were included at 12 weeks; (b) Body weight in each group over time (Model M005).

FIG. 5 (a) Number of mice that had metastases at sacrifice as detected macroscopically or evaluated by H&E staining; (b) Residual disease at week 12.

FIG. 6 (a) Mouse model M001: Injection frequency, dosing and site of injection during treatment period; (b) Change in mean tumor volume over time; (c) Dot plot showing tumor volume per mouse at 6 weeks.

FIG. 7 (a) Body weight in each group over time (Model M001), combination treatment was not toxic; (b) Treatment with a bispecific MF5816xMF3755 alone or a bispecific MF5816xMF3755+irinotecan blocked metastasis. Metastases were evaluated in tissues macroscopically and by staining with H&E.

FIG. 8 (a) Amino acid sequences of heavy chain variable regions of MF5816xMF3755 that together with a common light chain variable region such as the variable region of the human kappa light chain IgVκ1 39*01/IGJκ1*01 form a variable domain that binds LGR5 or EGFR. The CDR and framework regions are indicated in FIG. 8b. A DNA sequence is indicated in FIG. 8c. Additional heavy chain variable regions binding EGFR and LGR5, which are suitable for the generation of bispecific antibodies in combination with a topoisomerase I inhibitor are further disclosed in this figure.

FIG. 9 Amino acid sequence of a) a common light chain amino acid sequence. b) common light chain variable region DNA sequence and translation (IGKV1-39/jk1). c) Common light chain constant region DNA sequence and translation. d) IGKV1-39/jk5 common light chain variable region translation. e) V-region IGKV1-39A; f) CDR1, CDR2 and CDR3 of a common light chain.

FIG. 10. IgG heavy chains for the generation of bispecific molecules. a) CH1 region. b) hinge region. c) CH2 region. d) CH3 domain containing variations L351K and T366K (KK). e) CH3 domain containing variations L351D and L368E (DE).

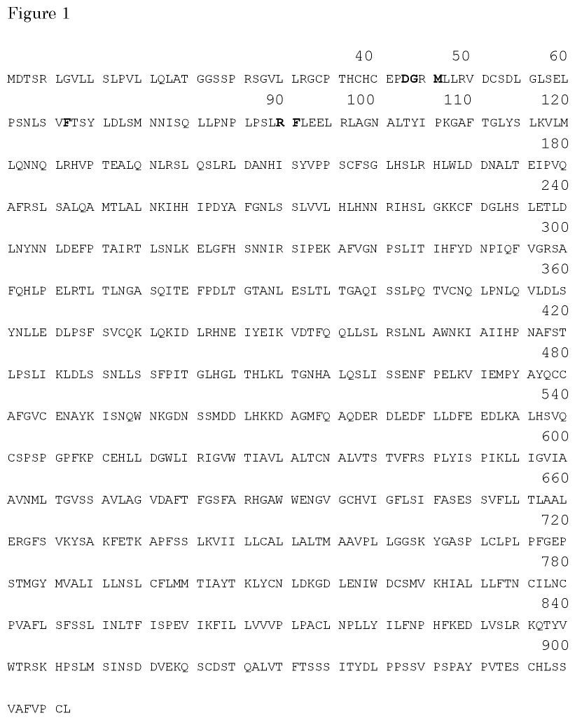

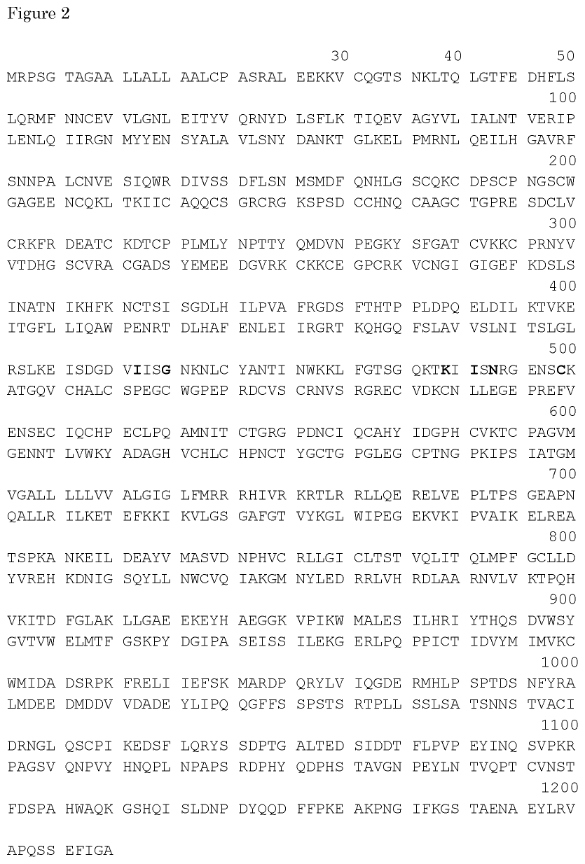

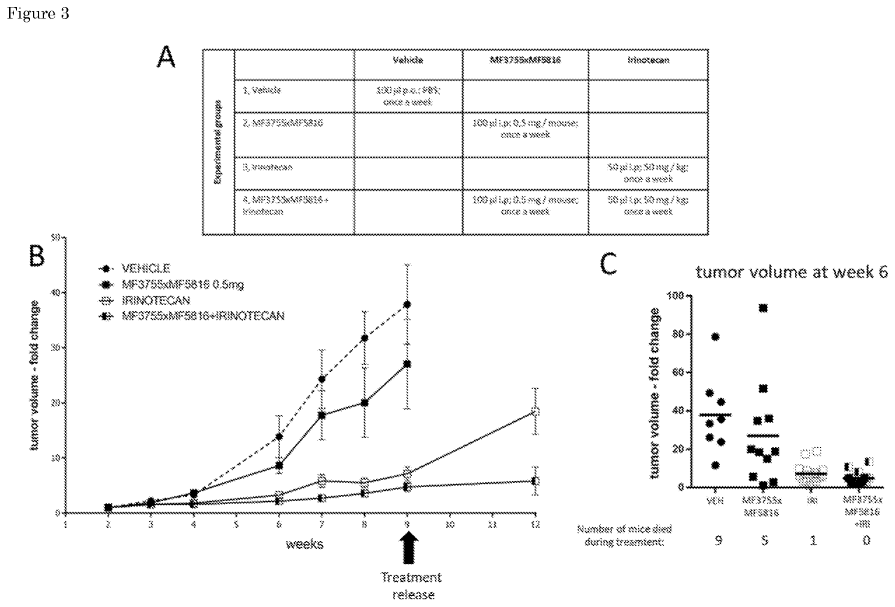

### EXAMPLES

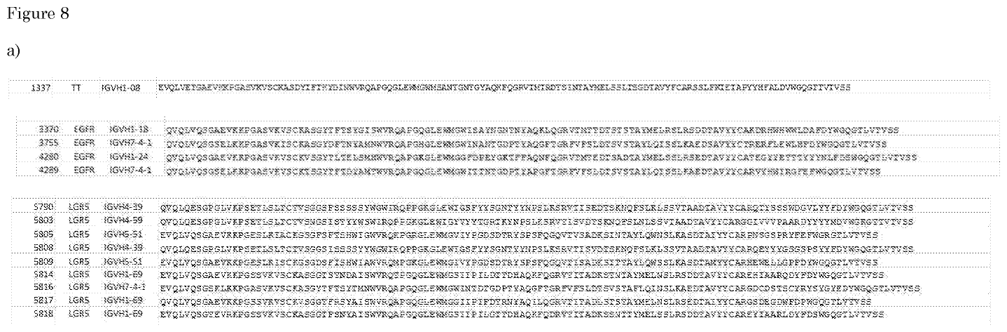

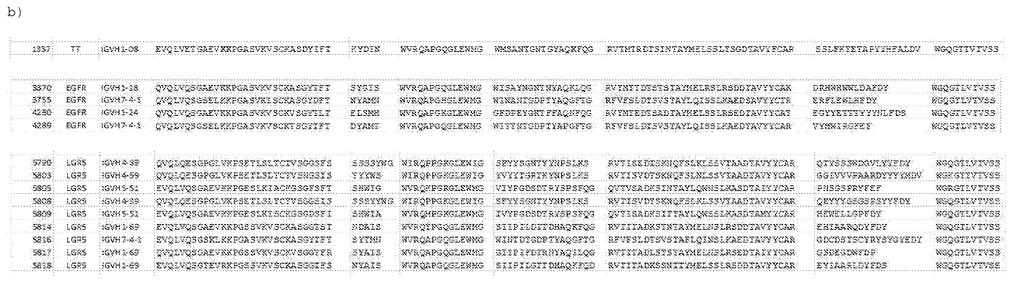

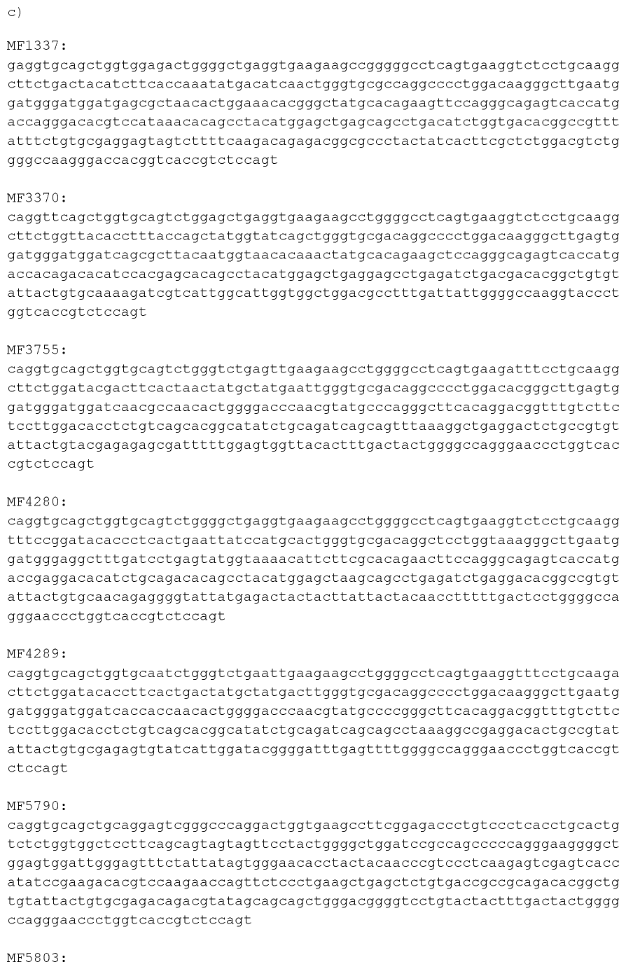

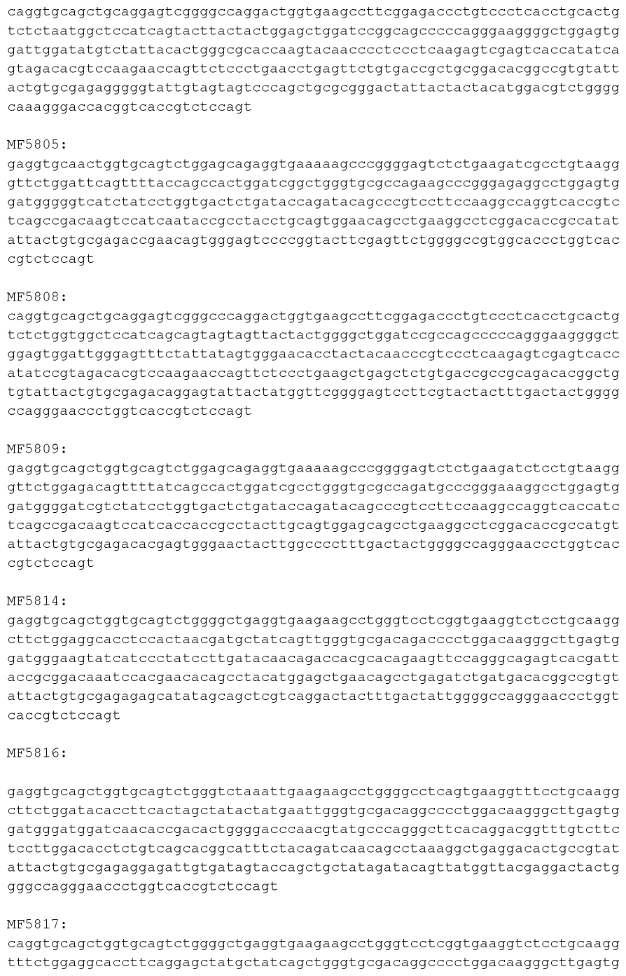

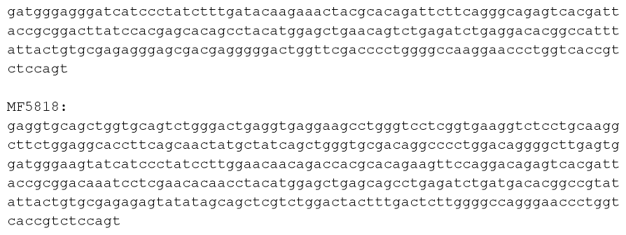

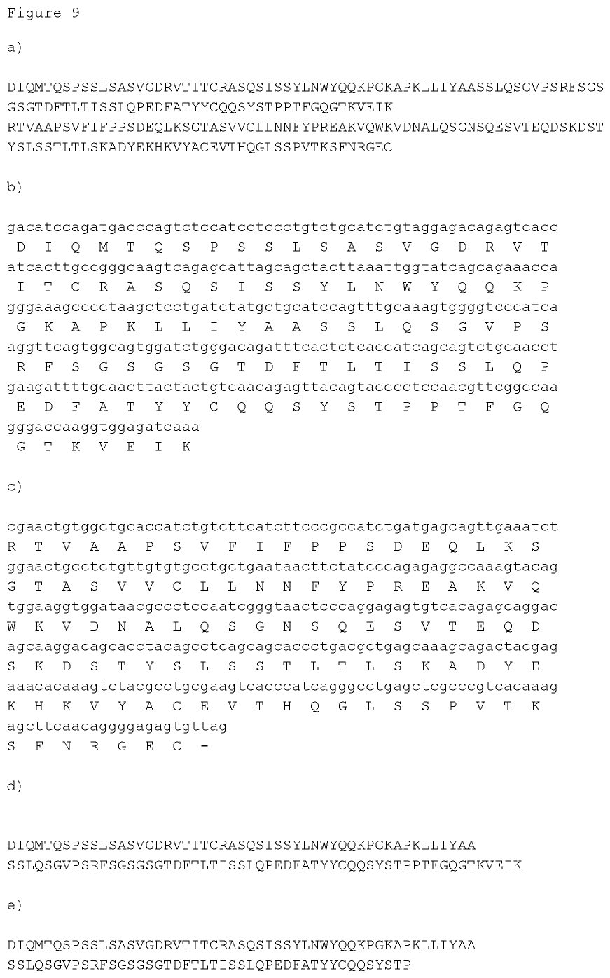

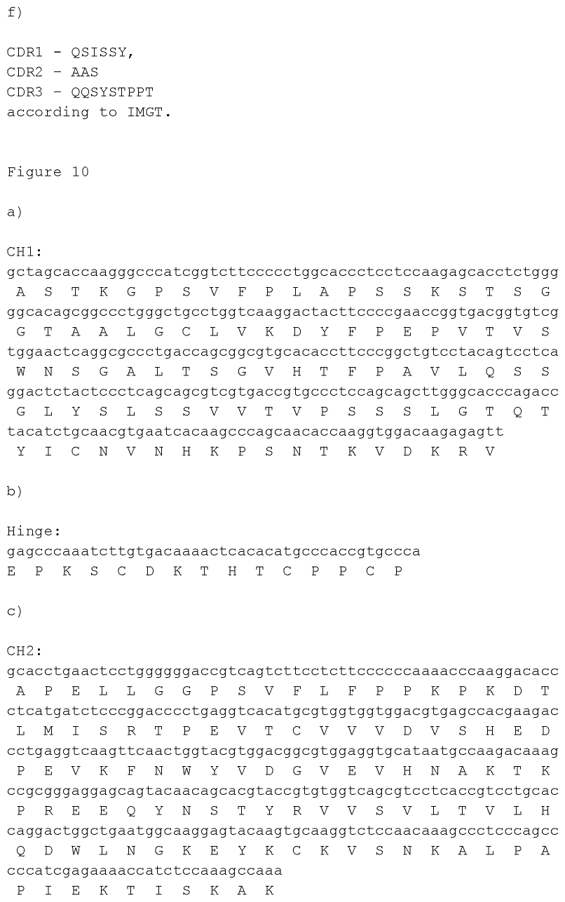

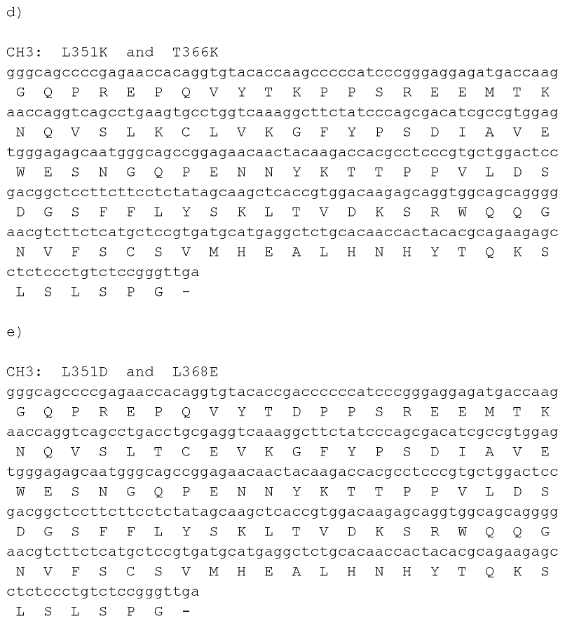

As used herein “MFXXXX” wherein X is independently a numeral 0-9, refers to a Fab comprising a variable domain wherein the VH has the amino acid sequence identified by the 4 digits depicted in FIG. 8. Unless otherwise indicated the light chain variable region of the variable domain typically has a sequence of FIG. 9b. The light chain in the examples has a sequence as depicted in FIG. 9a. “MFXXXX VH” refers to the amino acid sequence of the VH identified by the 4 digits. The MF further comprises a constant region of a light chain and a constant region of a heavy chain that normally interacts with a constant region of a light chain. The VH/variable region of the heavy chains differs and typically also the CH3 region, wherein one of the heavy chains has a KK mutation of its CH3 domain and the other has the complementing DE mutation of its CH3 domain (see for reference PCT/NL2013/050294 (published as WO2013/157954) and FIGS. 10d and 10e. Bispecific antibodies in the examples have an Fc tail with a KK/DE CH3 heterodimerization domain, a CH2 domain and a CH1 domain as indicated in FIG. 10, a common light chain as indicated in FIG. 9a and a VH as specified by the MF number. For example a bispecific antibody indicated by MF3755xMF5816 has the above general sequences and a variable domain with a VH with the sequence of MF3755 and a variable domain with a VH with the sequence of MF5816.

### Example 1

### Cell Lines

Freestyle 293F cells (cat. nr. p/n51-0029) were obtained from Invitrogen and routinely maintained in 293 FreeStyle medium. HEK293T (ATCC-CRL-11268) and CHO-K1 (DSMZ ACC110) cell lines were purchased from ATCC and routinely maintained in DMEM/F12 (Gibco) supplemented with L-Glutamine (Gibco) and FBS (Lonza).

The amino acid and nucleic acid sequences of the various heavy chain variable region (VH) are indicated in FIG. 8. Bispecific antibodies EGFR/LGR5, MF3755xMF5814; comprising heavy chain variable regions MF3755 and MF5816 and a common light chain and including modifications for enhanced ADCC from afucocylation, among other LGR5 and EGFR combinations as depicted in FIG. 9a have been shown to be effective in WO2017/069628 (page 138).

### Generation of Bispecific Antibodies

Bispecific antibodies were generated by transient co-transfection of two plasmids encoding IgG with different VH domains, using a proprietary CH3 engineering technology to ensure efficient heterodimerisation and formation of bispecific antibodies. The common light chain is also co-transfected in the same cell, either on the same plasmid or on another plasmid. In our applications (e.g. WO2013/157954 and WO2013/157953; incorporated herein by reference) we have disclosed methods and means for producing bispecific antibodies from a single cell, whereby means are provided that favor the formation of bispecific antibodies over the formation of monospecific antibodies. These methods can also be favorably employed in the present invention. Specifically, preferred mutations to produce essentially only bispecific full length IgG molecules are amino acid substitutions at positions 351 and 366, e.g. L351K and T366K (numbering according to EU numbering) in the first CH3 domain (the ‘KK-variant’ heavy chain) and amino acid substitutions at positions 351 and 368, e.g. L351D and L368E in the second CH3 domain (the ‘DE-variant’ heavy chain), or vice versa (see FIGS. 10d and 10e). It was previously demonstrated in the mentioned applications that the negatively charged DE-variant heavy chain and positively charged KK- variant heavy chain preferentially pair to form heterodimers (so-called ‘DEKK’ bispecific molecules). Homodimerization of DE-variant heavy chains (DE-DE homodimers) or KK-variant heavy chains (KK-KK homodimers) hardly occurs due to strong repulsion between the charged residues in the CH3-CH3 interface between identical heavy chains.

VH genes of variable domain that bind LGR5 described above were cloned into the vector encoding the positively charged CH3 domain. The VH genes of variable domain that bind EGFR such as those disclosed in WO 2015/130172 (incorporated herein by reference) were cloned into vector encoding the negatively charged CH3 domain. Suspension growth-adapted 293F Freestyle cells were cultivated in T125 flasks on a shaker plateau until a density of 3.0×10e6 cells/ml. Cells were seeded at a density of 0.3-0.5×10e6 viable cells/ml in each well of a 24-deep well plate. The cells were transiently transfected with a mix of two plasmids encoding different antibodies, cloned into the proprietary vector system. Seven days after transfection, the cellular supernatant was harvested and filtered through a 0.22 μM filter (Sartorius). The sterile supernatant was stored at 4° C. until purification of the antibodies.

### IgG Purification

Purifications were performed under sterile conditions in filter plates using filtration. First, the pH of the medium was adjusted to pH 8.0 and subsequently, IgG-containing supernatants were incubated with protein A Sepharose CL-4B beads (50% v/v) (Pierce) for 2 hrs at 25° C. on a shaking platform at 600 rpm. Next, the beads were harvested by filtration. Beads were washed twice with PBS pH 7.4. Bound IgG was then eluted at pH 3.0 with 0.1 M citrate buffer and the eluate was immediately neutralized using Tris pH 8.0. Buffer exchange was performed by centrifugation using multiscreen Ultracel 10 multiplates (Millipore). The samples were finally harvested in PBS pH7.4. The IgG concentration was measured using Octet. Protein samples were stored at 4° C.

### IgG Quantification using Octet

To determine the amount of IgG purified, the concentration of antibody was determined by means of Octet analysis using protein-A biosensors (Forte-Bio, according to the supplier's recommendations) using total human IgG (Sigma Aldrich, cat. nr. 14506) as standard.

### Mice and Preparation of Cells for Engraftment

Tumoroids were grown for seven days before disaggregating into a single cells suspension for injection. For all mouse studies female NOD.CB17/AlhnRj-Prkdcscid/Rj mice (Janvier Labs) aged between 6-8 weeks were used.

### Culture conditions and the Method of Creating Single Cells

Organoids derived from a colorectal cancer sample were cultured in 100% Basement Membrane Extracts (BME, Amsbio), at 37° C. and 5% CO2, with media composed of Advanced DMEM/F12 (Invitrogen) supplemented with: 2 mM GlutaMax (Invitrogen), 10 mM HEPES (Invitrogen), 1×B27 retinoic acid free (Invitrogen), 50 ng/mL EGF (Peprotech), 0.1 μg/mL Noggin (Peprotech), Rock-inhibiter Y-27632 (Sigma-Aldrich), 10 nM PGE2 (Sigma-Aldrich), 3 μm SB202190 (Sigma-Aldrich), 10 nM Gastrin (Tocris), 1 μg/ml R-SPO1 (home-made), 10 mM Nicotinomide (Sigma-Aldrich), 1.25 mM N-Acetyl-cysteine (Sigma-Aldrich), 0.5 μM A83-01 (Tocris). The day prior to analysis, the organoids were disaggregated into single cells. To this aim, the organoids were first liberated from the BME by removing the culturing media, and re-suspending the BME in cell recovery solution (BD Biosciences), and incubating for 1 hour on ice. Subsequently, the organoids were centrifuged (all centrifuge steps were for 5 minutes, 200 g at 4° C.). The pellet was re-suspended in 1 mL of 50% Trypsin/EDTA Solution (TE); 50% PBS, and pipetted up and down, and regularly visually assessed until a single cell suspension was achieved. The TE was diluted in 10 mL of PBS and centrifuged. The cells were washed twice in 10 mL of PBS before re-suspending in BME and aliquoting into 50 μL drops on to pre-warmed plates (37° C). The BME drops were left to set for 15 minutes before 500 μL of media were added per drop. After 12 hours the cells were isolated from the BME using cell recovery solution. After 1 hour on ice, the cells were centrifuged, and washed once in 10 mL of PBS containing 0.5% BSA and 0.5 mM EDTA (staining buffer). The pellet was then re-suspended in staining buffer and counted.

The Stem Cell and Cancer Group at VHIO has developed a collection of CRC PDX models derived from surgically resected primary tumors (colon and rectum) and liver metastases. PDX models are clinically and molecularly annotated and faithfully represent the clinical epidemiology of mCRC. These models can be injected subcutaneously or orthotopically in the cecum wall of immunodeficient mice. Orthotopic models generate local and distant metastases in lymph nodes, liver, lungs and carcinomatoses, reproducing the advance disease in CRC patients. A set of PDX models with key molecular traits was selected to evaluate the efficacy of anti-LGR5/EGFR bi-specific antibodies of the invention (See Table 1). Several mutant and wild type models were selected in the initial PDX set. Other determinants such as the relative expression of EGFR or LGR5 that could also determine response to the EGFR/LGR5 antibodies developed have been measured in these PDX models (Table 1).

PDX models derived from liver metastases of three advanced CRC patients (Table 1) were selected. Two models are KRAS mutant (G13D and G12D for M005 and M001, respectively), of which M005 is also an APC and a PIK3CA 112_112del mutant.

Model M005: 120 NOD-SCID mice were given orthotopic cecum wall injections of 1×10⁶ tumor cells derived from the M005 PDX model, where the model was generated essentially as described in Puig et al., A Personalized Preclinical Model to Evaluate the Metastatic Potential of Patient-Derived Colon Cancer Initiating Cells, Clin Cancer Res; 19 (24), 6787-6801 (2013), which is incorporated in its entirety into this application. These human tumor cells were derived from a CRC liver metastasis and contain mutations in the KRAS gene (KRAS G13) and in the PIK3CA gene (PIK3CA 112_112del). See UK Sanger Institute, featured 18 different tissue types carrying mutation PIK3C C420R (cancer.sanger.ac.uk/cosmic, mutation ID COSM757). From 15 days post-injection, weekly CT imaging was used to monitor mice and detect primary tumors in the cecum. Treatments were initiated after at least 80% of animals had a primary tumor growing in the cecum. The following 18 mice were excluded: which died after surgery (#5), with no primary tumor (#7), too small or too large tumors (#2 and #1 respectively), low body weight (#2), general signs of illness (#1).

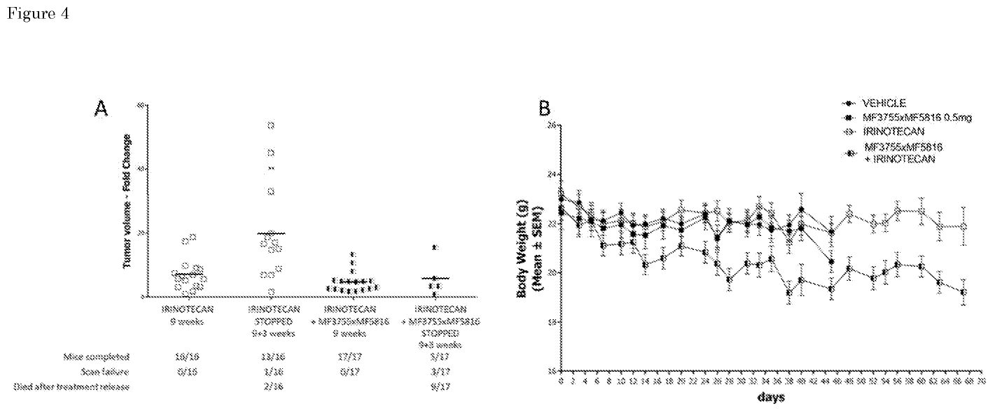

Remaining 102 mice were treated according to FIG. 3a and imaged weekly with microCT. The frequency and size of metastatic lesions was also determined by histological evaluation of liver and lungs (Hematoxylin and eosin stain (H&E) staining) Peritoneal carcinomatoses were detected macroscopically upon necropsy and later confirmed by histology.

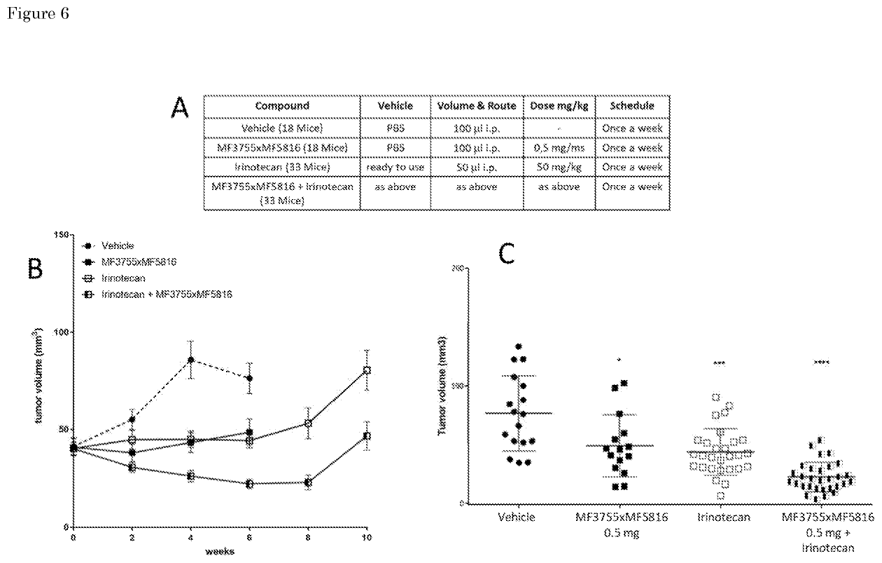

Model M001: M001 PDX model was generated essentially as described in Puig et al., A Personalized Preclinical Model to Evaluate the Metastatic Potential of Patient-Derived Colon Cancer Initiating Cells, Clin Cancer Res; 19 (24), 6787-6801 (2013), which is incorporated in its entirety into this application; See UK Sanger Institute, featured 18 different tissue types carrying mutation PIK3C C420R (cancer.sanger.ac.uk/cosmic, mutation ID COSM757). In the second orthotopic model, injected human tumor cells were originally derived from a CRC liver metastasis with mutations: KRAS G12D and PIK3CA-C420R. Injections of tumor cells were similarly done as above. The following 18 mice were excluded: which died after surgery (#11), with no primary tumor (#2), mice with too large tumors (#2), low body weight (#1), and general signs of illness (#2). Dosing and treatment regime were according to FIG. 6a.

At week 6, all mice treated with vehicle or the bispecific EGFR/LGR5, comprising MF3755 and MF5816 only were sacrificed; roughly half of the mice treated with irinotecan or the bispecific EGFR/LGR5, comprising MF3755 and MF5816+irinotecan were also sacrificed.

### Results

### Analysis Model M005

The mean tumor volume in mice treated with the bispecific EGFR/LGR5, comprising MF3755 and MF5816 alone was lower than mice given vehicle, but not as low as that of mice treated with irinotecan alone. Surprisingly, mice receiving the bispecific EGFR/LGR5, comprising MF3755 and MF5816+irinotecan combination treatment had a lower tumor volume compared to all other groups of mice (FIG. 3b, 3c). Interestingly, after treatment release, the bispecific EGFR/LGR5, comprising MF3755 and MF5816 prolonged the tumor growth-blocking effect of irinotecan as seen in the fold change in tumor volume (FIG. 4a).

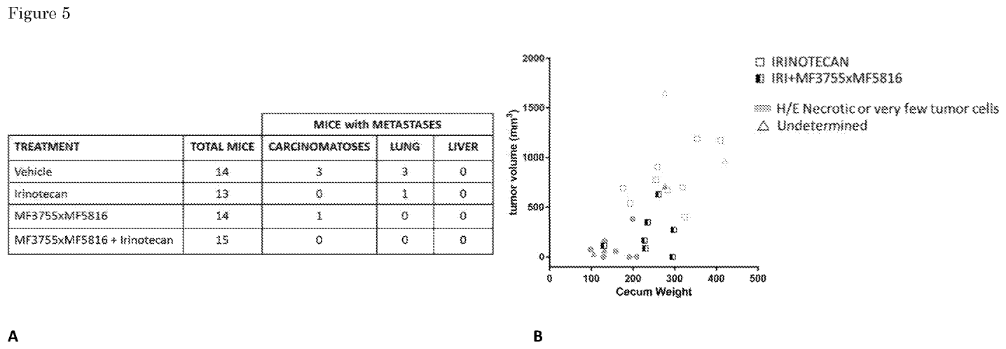

The primary tumors from all mice were harvested at sacrifice and analyzed for frequency and size of metastatic lesions. FIG. 5a shows the numbers of mice found to have metastatic lesions at sacrifice, demonstrating that mice treated with the bispecific EGFR/LGR5, comprising MF3755 and MF5816 or irinotecan, either alone or in combination, had fewer metastases than untreated mice.

Tissues were also analyzed in mice which were subjected to treatment release (9 weeks) and sacrifice after a 3-week treatment-free period. Smaller tumors were found to contain necrotic cells or only a small number of tumor cells, whereas most of the larger tumors had abundant tumor cells (FIG. 5b). This analysis showed that tumor volume and cecum weight were positively correlated in treated mice, 3 weeks after treatment release (P<0.0001 for Pearson correlation coefficient).

### Analysis Model M001

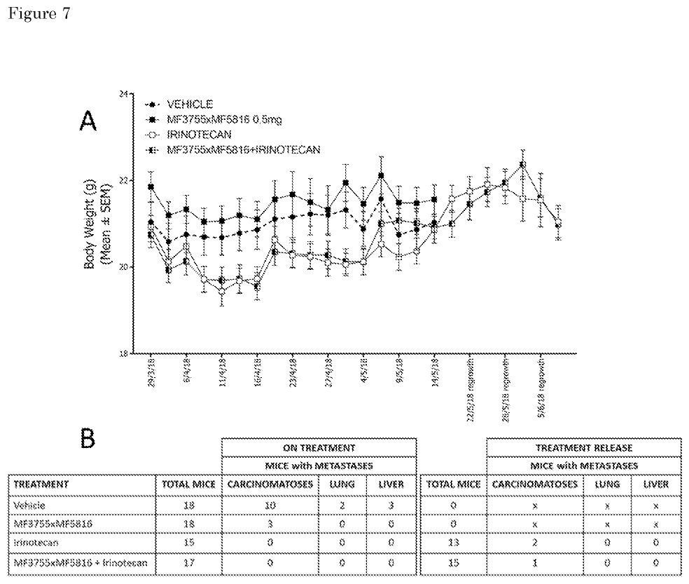

The mean tumor volume in mice treated with the bispecific EGFR/LGR5, comprising MF3755 and MF5816 alone was very similar to that of mice treated with irinotecan alone. However, mice receiving the bispecific EGFR/LGR5, comprising MF3755 and MF5816+irinotecan combination treatment had a lower tumor volume than any of the other group of mice (FIG. 6b,c). No toxicity was observed in mice receiving the combination of the bispecific EGFR/LGR5, comprising MF3755 and MF5816+irinotecan (FIG. 7a).

Histological analysis to determine metastatic lesions at sacrifice demonstrated that mice treated with bispecific EGFR/LGR5, comprising MF3755 and MF5816 or irinotecan, either alone or in combination, had fewer metastases than untreated mice (FIG. 7b).

Two orthotopic models M005 and M001 were tested with the bispecific antibody EGFR/LGR5, comprising MF3755 and MF5816 and chemotherapy drug irinotecan, alone and in combination for their potential to inhibit tumor growth and metastatic potential. In M005, the bispecific antibody EGFR/LGR5, comprising MF3755 and MF5816 and irinotecan alone were able to delay primary tumor growth, but combination of the two demonstrated the bispecific antibody EGFR/LGR5, comprising MF3755 and MF5816 and irinotecan promote a superior response. After treatment release, combined treatment completely eliminated primary tumors in five out of five surviving mice. For irinotecan monotherapy this was the case in just 1 out of 14 mice. Again, this indicates that the bispecific antibody EGFR/LGR5, comprising MF3755 and MF5816 potentiates complete tumor regression induced by chemotherapy. In terms of metastatic potential, the bispecific antibody EGFR/LGR5, comprising MF3755 and MF5816 blocked the formation of distant metastases, as did irinotecan. No metastases were seen in mice treated with combined irinotecan+the bispecific antibody EGFR/LGR5, comprising MF3755 and MF5816.

The results from model M005 were confirmed in model M001 model. The bispecific antibody EGFR/LGR5, comprising gMF3755 and MF5816 and irinotecan alone were equally effective at delaying primary tumor growth in M001, however, when administered together, the combined treatment appeared to be more effective than either agent given alone.

Statistical analysis (ANCOVA) on tumor volumes at week 6 of data shown in FIG. 6c showed that treatment significantly reduced tumor volume between all of the groups except between irinotecan and the bispecific antibody comprising MF3755 and MF5816. (Vehicle vs. MF3755 and MF5816, p<0.0001; vehicle vs. irinotecan, p<0.0001; vehicle vs. irinotecan+MF3755 and MF5816, p<0.0001; MF3755 and MF5816 vs. irinotecan, p<0.6429; MF3755 and MF5816 vs irinotecan+MF3755 and MF5816, p<0.0001; irinotecan+MF3755 and MF5816 vs. irinotecan, p<0.0001.)

In M001 combined treatment was not more toxic than irinotecan alone. In terms of metastatic potential, EGFR/LGR5, MF3755 and MF5816 blocked the formation of distant metastases, as did irinotecan. No metastases were seen in mice treated with combined irinotecan+the bispecific antibody EGFR/LGR5, comprising MF3755 and MF5816.

In conclusion, using two orthotopic CRC tumor models, it has been found that a combination treatment of the bispecific antibody EGFR/LGR5, comprising MF3755 and MF5816 and irinotecan results in tumor regression to a greater degree compared to when these drugs are administered alone. In addition, metastasis was found to be blocked in the bispecific antibody EGFR/LGR5, comprising MF3755 and MF5816 and irinotecan alone or combined treatments.

TABLE 1
Characteristics of PDX models originating from liver metastases of CRC patients. LGR5, EGFR and
nuclear β-catenin were determined by immunofluorescence quantification. Mutation status of
Wnt signaling (APC, RSPO, RNF43, ZNRF3) and oncogenic (KRAS, PIK3CA, TP53) proteins were determined
by genomic analysis. Sensitivity of the PDX models (grown subcutaneously) to WNT inhibitors is
indicated in the dark cells. The PDX model T108 was not used in the further experiments.

| PDX | | | Nuclear | | RSPO | | | | | | |
|---|---|---|---|---|---|---|---|---|---|---|---|
| ID | LGR5 | EGFR | β-cat | APC | FUSIONS | RNF43 | ZNRF3 | KRAS | PIK3CA | TP53 | MSI |
| M005 | 1398.06 | 348.29 | 10868 | MUT | WT | WT | WT | MUT G13D | MUT | WT | MSS |
| T108 | 4331.36 | NA | NA | WT | MUT | WT | WT | WT | WT | MUT | NA |
| M001 | 5757.52 | 483.81 | 2501 | WT | WT | WT | WT | MUT G12D | MUT | MUT | NA |

1-29. (canceled)

30. A method of treating cancer in a subject in need thereof, comprising administering to the subject a therapeutically effective amount of a topoisomerase I inhibitor and a bispecific antibody or antigen-binding fragments thereof that comprise a variable domain that binds an extracellular part of EGFR and a variable domain that binds an extracellular part of LGR5.

31. The method of claim 30, wherein the cancer is colorectal, pulmonary, gastrointestinal or ovarian cancer.

32. The method of claim 31, wherein the cancer is colorectal cancer.

33. The method of claim 30, wherein the bispecific antibody or antigen-binding fragments thereof and the topoisomerase I inhibitor are administered to the subject concurrently or sequentially.

34. The method of claim 30, wherein the bispecific antibody or antigen-binding fragment thereof is administered to the subject prior to the topoisomerase I inhibitor.

35. The method of claim 30, wherein the bispecific antibody or antigen-binding fragments thereof comprise one VH domain that binds EGFR and comprises the amino acid sequence of MF3755 as depicted in FIG. 8 or comprising not more than 5, 4, 3, 2, or 1 conservative amino acid substitutions; and one VH region that binds LGR5 comprises the amino acid sequence of MF5816 as depicted in FIG. 8, or comprising not more than 5, 4, 3, 2, or 1 conservative amino acid substitutions, and wherein the VH region binds antigen in association with a VL region.

36. The method of claim 30, wherein the bispecific antibody or antigen-binding fragment thereof comprises a common light chain.

37. The method of claim 36, wherein the common light chain comprises a germline IgVκ1-39 variable region V-segment.

38. The method of claim 36, wherein the common light chain comprises the kappa light chain V-segment IgVκ1-39*01.

39. The method of claim 30, wherein the bispecific antibody or antigen-binding fragment thereof comprises a light chain variable region comprising an amino acid sequence that is at least 90%, at least 95%, at least 97%, at least 98%, at least 99% identical or 100% identical to the amino acid sequence set forth in FIG. 9.

40. The method of claim 30, wherein the topoisomerase I inhibitor is camptothecin or a derivative thereof.

41. The method of claim 30, wherein the topoisomerase I inhibitor is irinotecan or topotecan.

42. The method of claim 30, wherein the bispecific antibody or antigen-binding fragments thereof is antibody-dependent cellular cytotoxicity (ADCC) enhanced.

43. The method of claim 30, wherein the bispecific antibody or antigen-binding fragments thereof is afucosylated.

44. A method for inhibiting proliferation of a cell that expresses EGFR and LGR5 comprising contacting the cell with a topoisomerase I inhibitor and a bispecific antibody or antigen-binding fragments thereof that comprise a variable domain that binds an extracellular part of EGFR and a variable domain that binds an extracellular part of LGR5.

45. A pharmaceutical composition comprising a topoisomerase I inhibitor and a bispecific antibody or antigen-binding fragments thereof that comprise a variable domain that binds an extracellular part of EGFR and a variable domain that binds an extracellular part of LGR5.

46. The pharmaceutical composition according to claim 45, wherein the topoisomerase I inhibitor and the bispecific antibody or antigen-binding fragments thereof are provided in a single formulation.

47. The pharmaceutical composition according to claim 45, wherein the topoisomerase I inhibitor and the bispecific antibody or antigen-binding fragments thereof are provided in separate formulations.

48. A kit comprising the pharmaceutical composition of claim 45.
